(19) United States
(12) Patent Application Publication
(10) Pub. No.: US 2019/0276897 A1
(43) Pub. Date: Sep. 12, 2019

(54) EGFR AND PTEN GENE ALTERATIONS PREDICTS SURVIVAL IN PATIENTS WITH BRAIN TUMOR

(71) Applicant: EUROPATH BIOSCIENCES, S.L., Barcelona (ES)

(72) Inventors: Michael J. Donovan, Barcelona (ES); Anna COLOMER VALERO, Barcelona (ES); Nadina ERILL SAGALES, Barcelona (ES); Isidre FERRER ABINZANDA, Barcelona (ES); Susana BOLUDA CASAS, Barcelona (ES)

(73) Assignee: EUROPATH BIOSCIENCES, S.L.

(21) Appl. No.: 16/290,236

(22) Filed: Mar. 1, 2019

Related U.S. Application Data
(63) Continuation of application No. 14/581,066, filed on Dec. 23, 2014, now abandoned.
Application No. 14/581,066 is a continuation of application No. 12/962,244, filed on Dec. 7, 2010, now abandoned.
(60) Provisional application No. 61/283,793, filed on Dec. 8, 2009.

(51) Int. Cl. C12Q 1/6886; A61K 31/517; A61K 31/4188

(52) U.S. Cl. CPC C12Q 1/6886; A61K 31/517; A61K 31/4188; C12Q 2600/118; C12Q 2600/156; C12Q 2600/158; C12Q 2600/106

(57) ABSTRACT

The invention relates to methods of predicting the clinical outcome of brain cancer patients based on the LOH levels of the PTEN gene and on the expression levels or the polysomy/amplification levels of EGFR gene in a sample from said patients.

### CROSS-REFERENCE TO RELATED APPLICATIONS

This application is a Continuation Application of U.S. patent application Ser. No. 14/581,066, filed Dec. 23, 2014. U.S. patent application Ser. No. 14/581,066 is a Continuation Application of U.S. patent application Ser. No. 12/962,244, filed Dec. 7, 2010, which in turn claims benefit of U.S. Provisional Application No. 61/283,793, filed Dec. 8, 2009. The disclosures of such US patent application and US provisional application are hereby incorporated herein by reference in their respective entireties, for all purposes.

### FIELD OF THE INVENTION

The invention relates to the fields of diagnostics and therapeutics, in particular to a method of providing personalized management to brain cancer patients based on the expression of certain genes in a sample from said patients, which certain serve as treatment targets.

### BACKGROUND OF THE INVENTION

Gliomas: Diagnosis and Disease Categorization

A glioma is a type of cancer that starts in the brain or spine. It is called a glioma because it arises from glial cells and/or its precursors. The most common site of gliomas is the brain. Gliomas are classified by cell type, grade, and location. Gliomas are named according to the specific type of cell they most closely resemble. The main types of gliomas are:
Ependymomas, gliomas derived from ependymal cells.
Astrocytomas, gliomas derived from astrocytes; the glioblastoma multiforme (GBM) is the most common astrocytoma.
Oligodendrogliomas, gliomas derived from oligodendrocytes.
Mixed gliomas, such as oligoastrocytomas, that contain cells from different types of glia.

Gliomas are further categorized according to their grade, which is determined by pathologic evaluation of the tumor. Thus we can distinguish between low-grade gliomas that are well-differentiated (not anaplastic), benign and portend a better prognosis for the patient; and high-grade gliomas, that are undifferentiated or anaplastic, malignant and carry a worse prognosis.

Of numerous grading systems in use, the most common is the World Health Organization (WHO) grading system for astrocytoma.

Treatment of Brain Gliomas

The treatment for brain gliomas depends on the location, the cell type and the grade of malignancy. Often, treatment is a combined approach, using surgery, radiation therapy, and chemotherapy. The radiation therapy is in the form of external beam radiation or the stereotactic approach using radiosurgery. Spinal cord tumors can be treated by surgery and radiation. Temozolomide is a chemotherapeutic drug that is able to cross the blood-brain barrier effectively and is being used in therapy. Despite these approaches most high grade glioma patients succumb to their disease. New therapeutic interventions to critical targets are needed to improve outcome in this patient population.

Glioblastoma Multiforme (GBM)

The glioblastoma multiforme (GBM, WHO grade IV) is a highly aggressive brain tumor presenting as one of two subtypes with distinct clinical histories and molecular profiles. The primary GBM presents acutely as a high-grade disease and the secondary GBM subtype evolves from the slow progression of a low-grade disease.

Brown et al. (J Clin Oncology. 2008, 5603-5609) describe a phase I/II trial of erlotinib combined with temozolomide in patients suffering GBM. These authors tried to correlate the response of the patients with several molecular markers like EGFR, PTEN, P53, etc., but failed to observe any correlation between survival and expression levels of said genes.

Mirimanoff et al. (J Clin Oncology. 2006, 2563-2569) describe a completed EORTC Phase III trial, where the MGMT promoter methylation was the strongest predictor for outcome and positive response to temozolomide.

Van den Bent et al. (J Clin Oncology. 2009, 27:1268-1274) describe a recent randomized Phase II EORTC trial. Patients with progressive GBM after prior radiotherapy were randomly assigned to either erlotinib or a control arm that received treatment with either temozolomide or carmustine (BCNU). The primary end point was 6-month progression-free survival (PFS). Tumor specimens obtained at first surgery were investigated for EGFR expression; EGFRvIII mutants; EGFR amplification; EGFR mutations in exons 18, 19, and 21; and pAkt. No clear biomarker associated with improved outcome to erlotinib was identified.

Smith et al. (J. National Cancer Institute. 2001, 1246-1256) describe methods for predicting the survival of patients with anaplastic astrocytoma and GBM. These authors identify that mutation of PTEN and EGFR amplification are independent prognostic markers for patients with anaplastic astrocytoma and EGFR amplification is a survival marker for older patients with GBM.

Umesh et al. (Clinical Neuropathology. Vol. 28—No. 5/2009 (362-372)) describe a method for predicting the patient outcome of glioma comprising the detection of EGFR amplification and PTEN LOH by immunohistochemistry. The conclusion described in said document is that EGFR amplification associated with LOH of the PTEN gene is a trend to poor survival.

Prados et al. (J Clin Oncology. 2009, 27(4):579-84) describe a Phase II trial which evaluated erlotinib plus temozolomide during and after radiation, patients treated with combination therapy (i.e., erlotinib and temozolomide) had better survival. In addition, the study also evaluated several biomarkers and found that methylation of the MGMT promoter along with PTEN expression was associated with improved survival.

However, there is still a need for further markers and markers combinations useful for predicting the clinical outcome of the glioma patients. A special area for diagnosis and prognosis is the study of the biopsy sample. An integrated approach able to better define patient outcome based on the most appropriate treatment using tumor profiles will be critical, offering in addition to the glioma patient a better quality of life.

### SUMMARY OF THE INVENTION

In an aspect, the invention relates to a method for predicting the clinical outcome of a subject suffering from glioma which comprises determining the expression level of EGFR or the polysomy/amplification levels of the EGFR locus on chromosome 7 and the LOH levels of the PTEN gene in a sample from the same subject, and comparing said expression level or the polysomy/amplification levels of the EGFR gene and the LOH level of the PTEN gene with standard reference values, wherein the LOH level of the PTEN gene is measured by PCR, by a hybridization-based assay, by sequencing, or by SNP analysis; and wherein a high LOH level of the PTEN gene with respect to said standard reference value and a high expression level and/or high levels of polysomy/amplification of the EGFR gene with respect to said standard reference values are indicative of a good clinical outcome of the subject.

In another aspect, the invention relates to a method for predicting the clinical outcome of a subject suffering from glioma that comprises determining the LOH level of the PTEN gene in a sample from the subject, and comparing said LOH level of PTEN gene with a standard reference value, wherein the LOH level of the PTEN gene is measured by PCR, by a hybridization-based assay, by sequencing, or by SNP analysis; and wherein a high LOH level of the PTEN gene with respect to said standard reference value, is indicative of a bad clinical outcome of the subject.

In another aspect, the invention relates to a kit comprising agents capable of specifically detecting the expression level and/or the polysomy/amplification of the EGFR gene and the LOH of the PTEN gene and, optionally, a reagent for detecting a housekeeping gene or the protein encoded by said housekeeping gene and/or a reagent for detecting the chromosomes 7 and 10, wherein the set of agents capable of specifically determining the LOH level of the PTEN gene comprises a pair of oligonucleotide primers suitable for amplifying a specific fragment of the PTEN gene and/or an optionally labeled oligonucleotide probe which selectively binds to a target polynucleotide sequence on the chromosome region of the PTEN gene and/or reagents suitable for performing a sequencing reaction and/or reagents for performing a SNP analysis.

In another aspect, the invention relates to the use of said kit for predicting the clinical outcome of a subject suffering from glioblastoma multiforme, wherein if said agents detect a high expression level and/or high levels of polysomy/amplification of EGFR gene and a high LOH level of the PTEN gene, with respect to standard reference values, in a sample from said subject then the clinical outcome of the subject is good.

In another aspect, the invention relates to the use of erlotinib and/or temozolomide in the manufacture of a medicament for the treatment of a glioma in a subject suffering from a glioma, wherein the medicament is for a subject having a high LOH level of the PTEN gene, as measured by PCR, by a hybridization-based assay, by sequencing, or by an SNP analysis, with respect to a standard reference value and high expression levels and/or high polysomy/amplification of the EGFR gene with respect to standard reference values.

Use of radiotherapy in a regime for the treatment of a glioma in a subject suffering from a glioma, wherein said subject has a high LOH level of the PTEN gene, as measured by PCR, by a hybridization-based assay, by a sequencing technology, or by a SNP analysis, with respect to a standard reference value and high expression levels and/or high polysomy/amplification of the EGFR gene with respect to standard reference values.

### BRIEF DESCRIPTION OF THE DRAWINGS

FIG. 1. Kaplan-Meier survival curve illustrating time from first surgery to death/end-of-follow-up of only (Pure) GBM patients, astrocytoma patients and oligodendroglioma patients.

FIG. 2. Kaplan-Meier curve estimating survival from first surgery to death/recurrence/progression or end-of-follow-up of only (Pure) GBM patients, astrocytoma patients and oligodendroglioma patients.

FIG. 3. Kaplan-Meier survival curve demonstrating stratification of patients in the glioblastoma multiforme (GBM), anaplastic astrocytoma and mixed group with PTEN LOH (p=0.04).

FIG. 4. Kaplan-Meier survival curve demonstrating stratification of patients in the glioblastoma multiforme (GBM), anaplastic astrocytoma and mixed group as having both EGFR AMP/HP (amplification—high polysomy) and PTEN LOH (p=0.034).

FIG. 5. Representative images of glioblastoma multiforme (GBM) specimens stained with H&E (Hematoxylin and Eosin) (A). Example of FISH experiments where AMP/HP EGFR DNA FISH (B) and monosomy PTEN (C) are illustrated.

### DETAILED DESCRIPTION OF THE INVENTION

In order to facilitate the understanding of the invention described in this patent application, the meaning of some terms and expressions in the context of the invention are explained below.

The term “subject” refers to a member of a mammal animal species, and includes, but is not limited thereto, domestic animals, primates and humans; the subject is preferably a human being, male or female, of any age or race. Alternatively, the term “individual” is also sometimes used in this description to refer to human beings.

The term “protein” refers to a molecular chain of amino acids, linked by covalent or non-covalent bonds. The term includes all the forms of post-translational modifications, for example, glycosylation, phosphorylation or acetylation.

The term “antibody” refers to a protein with the capacity to specifically bind to an antigen. The term antibody comprises recombinant antibodies, monoclonal antibodies, or polyclonal antibodies, intact, or fragments thereof which maintain the capacity to bind to the antigen, combibodies, etc., both human or humanised and of non-human origin.

The term “primer oligonucleotide”, as used in the present invention, refers to a nucleotide sequence, which is complementary to a nucleotide sequence of a selected gene. Each primer oligonucleotide hybridises with its target nucleotide sequence and acts as an initiation point for DNA polymerisation.

The inventors of the present invention have found that the clinical outcome of patients suffering from glioma cancer correlates with expression levels and/or the polysomy/amplification levels of the EGFR gene and with the LOH level of the PTEN gene.

Thus, in an aspect, the invention relates to a method for predicting the clinical outcome of a subject suffering from glioma, hereinafter referred to as method [1] of the invention, that comprises:
a) determining the expression level or the polysomy/amplification level of the EGFR gene and the LOH level of the PTEN gene in a sample from the same subject, and
b) comparing said expression level or the polysomy/amplification level of the EGFR gene and the LOH level of the PTEN gene with standard reference values,
   wherein the LOH level of the PTEN gene is measured by PCR, by a hybridization-based assay, by sequencing, or by a SNP analysis; and
   wherein a high LOH level of the PTEN gene with respect to said standard reference value and a high expression level and/or high level of polysomy/amplification of the EGFR gene with respect to said standard reference values are indicative of a good clinical outcome of the subject.

In the present invention a “glioma” is a type of cancer that starts in the brain or spine. It is called a glioma because it arises from glial cells and/or its precursors. The most common site of gliomas is the brain. Gliomas are classified by cell type, grade, and location. Gliomas are named according to the specific type of cell they most closely resemble. The main types of gliomas are:
Ependymomas, gliomas derived from ependymal cells.
Astrocytomas, gliomas derived from astrocytes; the glioblastoma multiforme (GBM) is the most common astrocytoma.
Oligodendrogliomas, gliomas derived from oligodendrocytes.
Mixed gliomas, such as oligoastrocytomas, that contain cells from different types of glia.

Gliomas are further categorized according to their grade, which is determined by pathologic evaluation of the tumor. Thus, one can distinguish between (i) low-grade gliomas that are well-differentiated (not anaplastic), benign and portend a better prognosis for the patient; and (ii) high-grade gliomas, that are undifferentiated or anaplastic, malignant and carry a worse prognosis.

In a preferred embodiment, the glioma is a glioblastoma multiforme (GBM) and more preferably the glioblastoma is an early glioblastoma.

The glioblastoma multiforme (GBM) is the most common and malignant form of glial tumors and is composed of a heterogenous mixture of poorly differentiated malignant astrocytes and dysplastic endothelial cells. It primarily affects adults, involves the cerebral hemispheres and has a rapid disease course which often leads to death.

In the present invention “clinical outcome” is understood as the expected course of a disease. It denotes the doctor's prediction of how a subject's disease will progress, and whether there is chance of recovery or recurrence. The prediction of the clinical outcome can be done by using any endpoint measurements used in oncology and known to the skilled practitioner. Useful endpoint parameters to describe the evolution of a disease include:
disease-free progression which, as used herein, describes the proportion of patients in complete remission who have had no recurrence of disease during the time period under study;
objective response, which, as used herein, describes the proportion of treated people in whom a complete or partial response is observed;
tumor control, which, as used herein, relates to the proportion of treated people in whom complete response, partial response, minor response or stable disease in equal to or more than (≥) 6 months is observed;
progression free survival which, as used herein, is defined as the time from start of treatment to the first measurement of cancer growth;
six-month progression free survival or “PFS6” rate which, as used herein, relates to the percentage of people wherein free of progression in the first six months after the initiation of the therapy; and
median survival which, as used herein, relates to the time at which half of the patients enrolled in the study are still alive.

A good clinical outcome is understood as a situation where at least 10%, 20%, 30%, 40%, 50%, 60%, 70%, 80%, 90%, 95% or even more of the patients have a positive result regarding the endpoint parameters described above.

The term “sample” as used herein, relates to any sample which can be obtained from the patient. The present method can be applied to any type of biological sample from a patient, such as a biopsy sample, tissue, cell or fluid (whole blood, serum, saliva, semen, sputum, urine, cerebral spinal fluid (CSF), tears, mucus, sweat, milk, brain extracts and the like). In a particular embodiment, said sample is a tumour tissue sample or portion thereof. In a more particular embodiment, said tumor tissue sample is a brain tumor tissue sample from a patient suffering from brain cancer. Said sample can be obtained by conventional methods, e.g., biopsy, by using methods well known to those of ordinary skill in the related medical arts. Methods for obtaining the sample from the biopsy include gross apportioning of a mass, or microdissection or other art-known cell-separation methods. Tumour cells can additionally be obtained from fine needle aspiration cytology. In order to simplify conservation and handling of the samples, these can be formalin-fixed and paraffin-embedded or first frozen and then embedded in a cryosolidifiable medium, such as OCT-Compound, through immersion in a highly cryogenic medium that allows for rapid freeze. In a particular embodiment, the sample is a tumor sample that contains a substantially number of tumor cells.

The samples may be obtained from subjects previously diagnosed with glioma (patients), or from subjects who have not been previously diagnosed with glioma, or from patients diagnosed with glioma who are undergoing treatment, or from patients diagnosed with glioma who have been previously treated.

PTEN is the phosphatase and tensin homolog protein also known as BZS; MHAM; TEP1; MMAC1; PTEN1; 10q23del or MGC11227. PTEN is a protein, which in humans is encoded by the PTEN gene (RefSEq ID NM_000314 SEQ ID NO: 1, Protein reference NP_000305.3 SEQ ID NO: 2) (Steck P A, et al. 1997 Nat. Genet. 15 (4): 356-62 2). PTEN acts as a tumor suppressor gene through the action of its phosphatase protein product. This phosphatase is involved in the regulation of the cell cycle, preventing cells from growing and dividing too rapidly. Mutations of this gene contribute to the development of certain cancers (Chu E C, et al. 2004 Med. Sci. Monit. 10 (10): RA235-41 3). It does exist as homologues in other species, such as mice (NM_008960.2, SEQ ID NO: 3), rat (NM_031606.1, SEQ ID NO: 4), dog (NM_001003192, SEQ ID NO: 5), etc.

The protein encoded by the PTEN gene is a phosphatidylinositol-3,4,5-trisphosphate-3-phosphatase. It contains a tensin like domain as well as a catalytic domain similar to that of the dual specificity protein tyrosine phosphatases. Unlike most of the protein tyrosine phosphatases, this protein preferentially dephosphorylates phosphoinositide substrates. It negatively regulates intracellular levels of phosphatidylinositol-3,4,5-trisphosphate in cells and functions as a tumor suppressor by negatively regulating Akt/PKB signaling pathway (Hamada K, et al 2005 Genes Dev 19 (17): 2054-65).

The epidermal growth factor receptor (EGFR; ErbB-1; HER1 in humans) is the cell-surface receptor for members of the epidermal growth factor family (EGF-family) of extracellular protein ligands (Herbst R S (2004). Int. J. Radiat. Oncol. Biol. Phys. 59 (2 Suppl): 21-6.1) The EGFR is a member of the ErbB family of receptors. The EGFR is a protein which in humans is encoded by different isoforms: EGFR transcript variant 1 (NM_005228.3, SEQ ID NO: 6), transcript variant 2 (NM_201282.1, SEQ ID NO: 7), transcript variant 3 (NM_201283.1, SEQ ID NO: 8) and transcript variant 4 (NM_201284.1, SEQ ID NO: 9). It does exist as homologues in other species, such as mice (NM_207655.2, SEQ ID NO: 10 and NM_007912.4, SEQ ID NO: 11), rat (NM_031507.1, SEQ ID NO: 12), dog (XM_533073.2, SEQ ID NO: 13), etc.

The method [1] of the invention comprises determining the expression level of the EGFR gene and PTEN gene. As the person skilled in the art understands, the expression level of a gene can be determined by measuring the levels of mRNA encoded by said gene, by measuring both the levels of proteins encoded by said gene and the levels of variants thereof, by the use of surrogates (DNA copy number) for associating gene level with mRNA and protein product of said gene, etc.

A variant of a protein, e.g., EGFR or PTEN, as used herein may be (i) a protein in which one or more of the amino acid residues is/are substituted with a conserved or non-conserved amino acid residue (preferably a conserved amino acid residue) and such substituted amino acid residue may or may not be one encoded by the genetic code, (ii) a protein having one or more modified amino acid residues, e.g., residues that are modified by the attachment of substituent groups, (iii) a modified protein said protein being the results of an alternative splicing of the mRNA encoding the EGFR or PTEN protein, and/or (iv) a fragment of the protein. The term “fragment” includes also a peptide or protein generated via proteolytic cleavage (including multi-site proteolysis) of an original protein. Variants are deemed to be within the scope of those skilled in the art from the teaching herein.

As known in the art the “similarity” between two proteins is determined by comparing the amino acid sequence and its conserved amino acid substitutes of one protein to a sequence of a second protein. Variants according to the present invention include peptides or protein having amino acid sequences that are at least 60%, 65%, 70%, 72%, 74%, 76%, 78%, 80%, 82%, 84%, 86%, 88%, 90%, 95%, or even more, similar or identical to the original amino acid sequence. The degree of identity between two proteins can be determined using computer algorithms and methods that are widely known for the persons skilled in the art. The identity between two amino acid sequences is preferably determined by using the BLASTP algorithm (BLASTManual, Altschul, S., et al., NCBI NLM NIH Bethesda, Md. 20894, Altschul, S., et al., J. Mol. Biol. 215: 403-410 (1990)).

The proteins can be post-translationally modified. For example, post-translational modifications that fall within the scope of the present invention include signal peptide cleavage, glycosylation, acetylation, isoprenylation, proteolysis myristoylation, protein folding and proteolytic processing, etc. Additionally, the proteins may include unnatural amino acids formed by post-translational modification or by introducing unnatural amino acids during translation.

In a preferred embodiment, the determination of the expression levels of the EGFR gene and the PTEN gene can be carried out by measuring the expression level of the mRNA encoded by the EGFR gene or by the PTEN gene, respectively. For this purpose, the biological sample may be treated to physically or mechanically disrupt tissue or cell structure, to release intracellular components into an aqueous or organic solution to prepare nucleic acids for further analysis. The nucleic acids are extracted from the sample by procedures known to the skilled person and commercially available. RNA is then extracted from frozen or fresh samples by any of the methods known in the art, for example, Sambrook, Fischer and Maniatis, Molecular Cloning, a laboratory manual, (2nd ed.), Cold Spring Harbor Laboratory Press, New York, (1989). Preferably, care is taken to avoid degradation of the RNA during the extraction process.

In a particular embodiment, the expression level is determined by using mRNA obtained from a formalin-fixed, paraffin-embedded tissue sample. mRNA may be isolated from an archival pathological sample or biopsy sample which is first deparaffinized. An exemplary deparaffinization method involves washing the paraffinized sample with an organic solvent, such as xylene, for example. Deparaffinized samples can be rehydrated with an aqueous solution of a lower alcohol. Suitable lower alcohols, for example include, methanol, ethanol, propanols, and butanols. Deparaffinized samples may be rehydrated with successive washes with lower alcoholic solutions of decreasing concentration, for example. Alternatively, the sample is simultaneously deparaffinized and rehydrated. The sample is then lysed and RNA is extracted from the sample.

While all techniques of gene expression profiling (RT-PCR, SAGE, or TaqMan) are suitable for use in performing the foregoing aspects of the invention, the expression levels of the mRNA coding for EGFR or for PTEN are often determined by reverse transcription polymerase chain reaction (RT-PCR). The detection can be carried out in individual samples or in tissue microarrays.

In order to normalize the values of the mRNA expression among the different samples, it is possible to compare the expression levels of the mRNA of interest in the test samples with the expression of a control RNA. A “control RNA” as used herein, relates to a RNA whose expression levels do not change or change only in limited amounts in tumor cells with respect to non-tumorigenic cells. Preferably, a control RNA is a mRNA derived from a housekeeping gene and which code for a protein which is constitutively expressed and carry out essential cellular functions. Preferred housekeeping genes for use in the present invention include β-2-microglobulin, ubiquitin, 18-S ribosomal protein, cyclophilin, GAPDH and actin. In a preferred embodiment, the control RNA is β-actin mRNA. In an embodiment relative gene expression quantification is calculated according to the comparative Ct method using β-actin as an endogenous control and commercial RNA controls as calibrators. Final results are determined according to the formula 2−(ΔCt sample−ΔCt calibrator), wherein ΔCT values of the calibrator and sample are determined by subtracting the CT value of the target gene from the value of the β-actin gene.

The determination of the level of expression of the EGFR gene and PTEN gene needs to be correlated with the standard reference values. Standard reference values correspond to the median value of the expression levels of the EGFR gene and PTEN gene measured in a collection of samples from healthy patients. Once this median value is established, the level of this marker expressed in tumor tissues from patients can be compared with this median value, and thus be assigned a level of “low”, “normal” or “high”. The collection of samples from which the reference level is derived will preferably be constituted from healthy persons from the same age as the patients. In any case it can contain a different number of samples. In a more preferred embodiment, the samples are biopsy brain samples. Preferably the collection should be sufficient to provide an accurate standard reference value. Typically, the number of samples used for determining a standard reference value is at least 10, preferably more than 10, 20, 30, 40, 50, 60, 70, 80, 90, 100, 150, 200, 500 or even more samples. The standard can also include ‘normal’ cells present within the diseased and/or cancerous tissue. This is particularly true for brain tumor resection and occasionally biopsy specimens which typically contain a rim of normal tissue or have inflammatory cells etc, which do not contain gene changes. In addition, it can be used a cell culture system to evaluate gene content, as a way to determine the presence or loss of individual genes.

In a particular embodiment, an increase in the expression of the EGFR gene, or in the expression of the PTEN gene, as determined in the sample above the standard reference value of at least 1.1-fold, 1.5-fold, 5-fold, 10-fold, 20-fold, 30-fold, 40-fold, 50-fold, 60-fold, 70-fold, 80-fold, 90-fold, 100-fold or even more compared with the reference value is considered as a “high” expression level of the EGFR gene or as a “high” expression level of the PTEN gene, respectively. In another particular embodiment, a decrease in the expression of the EGFR gene, or in the expression of the PTEN gene, as determined in the sample below the standard reference value of at least 0.9-fold, 0.75-fold, 0.2-fold, 0.1-fold, 0.05-fold, 0.025-fold, 0.02-fold, 0.01-fold, 0.005-fold or even less compared with the standard reference value is considered as a “low” expression level of the EGFR gene, or as a “low” expression level of the PTEN gene, respectively.

Alternatively, in another particular embodiment, the expression level of the EGFR gene can be determined by measuring both the level of the protein encoded by said gene, i.e. EGFR protein, and the levels of a variant thereof. Although it would also be possible to determine the expression level of the PTEN gene by measuring both the level of the protein encoded by said gene, i.e. PTEN protein, and the levels of a variant thereof, in practice, said option is not suitable for performing the teachings of the instant invention as will be discussed below. The skilled person in the art knows that loss of protein expression may also be the result of methylation of the gene promoter and not a loss of the gene per se which impacts on other associated genes.

The determination of the expression level of a protein, e.g., EGFR protein, or a variant thereof can be carried out by any conventional technique known for the skilled person in the art. In a particular embodiment, the determination of the expression levels of said protein, e.g., EGFR protein, or a variant thereof, is carried out by immunological techniques such as e.g., ELISA (Enzyma-Linked ImmuneSorbent Assay), Western blot, immunofluorescence (IF), immunohistochemistry (IHC) analysis, etc. ELISA is based on the use of antigens or antibodies labeled (e.g., with enzymes) so that the conjugates formed between the target antigen and the labeled antibody results in the formation of, e.g., enzymatically-active complexes. Since one of the components (the antigen or the labelled antibody) is immobilised on a support, the antibody-antigen complexes are immobilised on the support and thus, it can be detected by the addition of a substrate which is converted by the enzyme to a product which is detectable by, e.g. spectrophotometry, fluorometry, etc. This technique does not allow the exact localisation of the target protein or the determination of its molecular weight but allows a very specific and highly sensitive detection of the target protein in a variety of biological samples (serum, plasma, tissue homogenates, postnuclear supernatants, ascites and the like). Western blot is based on the detection of a protein previously resolved by gel electrophoresis under denaturing conditions and immobilized on a membrane, generally nitrocellulose, by incubation with an antibody specific to said protein and a developing system (e.g. chemo-luminiscent, etc.). The analysis by immunofluorescence (IF) requires the use of an antibody specific for the target protein for the analysis of the expression and subcellular localization by microscopy. Generally, the cells under study are previously fixed with paraformaldehyde and permeabilised with a non-ionic detergent. In a preferred embodiment, the EGFR protein is detected by an immunohistochemistry (IHC) analysis using thin sections of the biological sample immobilised on coated slides. The sections are then deparaffinised, if derived from a paraffinised tissue sample, and treated so as to retrieve the antigen. The detection can be carried out in individual samples or in tissue microarrays. In another embodiment the method used for determining the expression level of a protein, e.g., EGFR protein, or a variant thereof is a proteomic array.

Practically any antibody or reagent known to bind with high affinity to the target protein can be used for detecting the amount of said target protein. It is preferred nevertheless the use of an antibody, for example polyclonal sera, hybridoma supernatants or monoclonal antibodies, antibody fragments, Fv, Fab, Fab′ y F(ab′)2, ScFv, diabodies, triabodies, tetrabodies and humanised antibodies.

In yet another embodiment, the determination of the expression level of a protein, e.g., EGFR, etc., can be carried out by constructing a tissue microarray (TMA) containing the patient samples assembled, and determining the expression levels of said protein by IHC techniques. Immunostaining intensity can be evaluated by two different pathologists and scored using uniform and clear cut-off criteria, in order to maintain the reproducibility of the method. Discrepancies can be resolved by simultaneous re-evaluation. Briefly, the result of immunostaining can be recorded as negative expression (0) versus positive expression, as low expression (1+) versus moderate (2+) expression and as high (3+) expression, taking into account the expression in tumoral cells and the specific cut-off for each marker. As a general criterion, the cut-offs were selected in order to facilitate reproducibility, and when possible, to translate biological events.

The determination of the expression level of the EGFR gene and PTEN gene needs to be correlated with the standard reference values which correspond to the median value of expression levels of the EGFR gene and PTEN gene measured in a collection of brain tissue samples from healthy patients. Preferably the collection should be sufficient to provide an accurate reference level. Typically the number of samples used for determining the standard reference values is at least 10, preferably more than 10, 20, 30, 40, 50, 60, 70, 80, 90, 100, 150, 200, 500, or even more, samples.

In a preferred embodiment the sample is a biopsy. Once this median value is established, the level of this marker expressed in tumor tissues from patients can be compared with this median value, and thus be assigned a level of “low”, “normal” or “high” as defined above.

“Polysomy”/“amplification” of the EGFR locus on chromosome 7 as used in the present invention is understood as the presence of more than one copy of the EGFR locus on the chromosome 7.

Further, as used herein, the term “LOH” within the context of the present invention refers to “loss of heterozygosity”. LOH in a cell represents the loss of normal function of one allele of a gene in which the other allele was already inactivated. This term is mostly used in the context of oncogenesis; after an inactivating mutation in one allele of a tumor suppressor gene occurs in the parent's germline cell, it is passed on to the zygote resulting in an offspring that is heterozygous for that allele. In oncology, loss of heterozygosity (LOH) occurs when the remaining functional allele in a somatic cell of the offspring becomes inactivated by mutation. In the case of PTEN LOH, this results in no normal tumor suppressor being produced.

In a particular embodiment, the determination of the polysomy/amplification level of the EGFR gene and the determination of the LOH levels of the PTEN gene can be measured, for example, in the DNA obtained from the tumor cells according to standard procedures such as, for example, quantitative PCR, comparative genomic hybridization (CGH) to microarray technologies, etc.; or in the tumor cells from the paraffinized-embedded section or from the cytology preparation by FISH using appropriate molecular probes, etc.

In a particular embodiment, the detection of the polysomy/amplification level of the EGFR gene and the determination of the LOH levels of the PTEN gene is carried out by a polymerase chain reaction (PCR).

In a particular embodiment, the detection of the polysomy/amplification level of the EGFR gene and the determination of the LOH levels of the PTEN gene is carried out by a hybridization-based assay. In a particular embodiment, the detecting step of the method [1] of the invention comprises contacting the nucleic acid sample with one or more nucleic acid probes each of which selectively binds to a target polynucleotide sequence on the chromosome region of the EGFR or PTEN loci, under conditions in which the probe forms a stable hybridization complex with the target polynucleotide sequence; and detecting the hybridization complex. In a particular embodiment, the nucleic acid probes used in the method [1] of the invention are labeled with a fluorophore. Alternatively, in another particular embodiment, the step of detecting the hybridization complex comprises determining the copy number of the target polynucleotide sequence, thereby determining the presence of polysomy or LOH of the target gene.

In a preferred embodiment, said hybridization-based assay is selected from the group consisting of Southern blot, in situ hybridization (ISH), fluorescence ISH (FISH) and comparative genomic hybridization (CGH). Southern blot is a method routinely used in molecular biology for detection of a specific DNA sequence in DNA samples; Southern blotting generally combines transfer of electrophoresis-separated DNA fragments to a filter membrane and subsequent fragment detection by probe hybridization. In situ hybridization (ISH) is a type of hybridization that uses a labeled complementary DNA or RNA strand (i.e., probe) to localize a specific DNA or RNA sequence in a portion or section of tissue (in situ), or, if the tissue is small enough, in the entire tissue; DNA ISH can be used to determine the structure of chromosomes. Fluorescence in situ hybridization (FISH) is a cytogenetic technique used to detect and localize the presence or absence of specific DNA sequences on chromosomes. FISH uses fluorescent probes that bind to only those parts of the chromosome with which they show a high degree of sequence similarity. Fluorescence microscopy can be used to find out where the fluorescent probe bound to the chromosomes. FISH is often used for finding specific features in DNA for use in genetic counselling, medicine (for example, in medical diagnostics to assess chromosomal integrity), and species identification; FISH can also be used to detect and localize specific mRNAs and other transcripts within tissue sections or whole mounts (so, it can help define the spatial-temporal patterns of gene expression within cells and tissues). Comparative genomic hybridization (CGH) or Chromosomal Microarray Analysis (CMA) is a molecular-cytogenetic method for the analysis of copy number changes (gains/losses) in the DNA content of a given subject's DNA and often in tumor cells; CGH detects only unbalanced chromosomal changes. In a particularly preferred embodiment, said hybridization-based assay is a CGH assay.

In a particular embodiment, said hybridization-based assay is an array-based assay. In a particular embodiment, once the sample has been obtained and the total DNA has been extracted, genome-wide analysis of DNA copy number changes by CGH is carried out. In general, for a typical CGH measurement, total genomic DNA is isolated from test and reference cell populations, differentially labeled and hybridized to a representation of the genome that allows the binding of sequences at different genomic locations to be distinguished. Hybridization reactions can be performed under conditions of different stringency. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions.

The amount of specimen DNA is frequently a constraint on CGH measurements. Typical array CGH procedures use from 300 ng to 3 μg of specimen DNA in the labeling reaction, equivalent to approximately 50,000 to 500,000 mammalian cells. Usually, random primer labeling protocols are employed, which also amplify the DNA, so that several micrograms (μg) are used in the hybridization.

Array CGH has been implemented using a wide variety of techniques. In a particular embodiment, array CGH is carried out using arrays from large-insert genomic clones such as bacterial artificial chromosomes (BACs). The general principles and conditions for detection of nucleic acids, such as using array CGH to BAC microarrays are well known for the skilled person in the art. This technique allows scanning the entire genome for DNA copy number changes therefore allowing quantitative detection of DNA copy number variation in tumor genomes with high resolution (Pinkel D, et al. Nat Genet 1998; 20(2):207-11; Hodgson G, et al. Nat Genet 2001; 29(4):459-64). As an illustrative non-limitative example, in the array CGH carried out by the method [1] of the invention test tumor and reference genomic DNAs can be labeled by random priming using Cy3 and Cy5 fluorophores. Then, the images of the arrays may be analysed using, for example, a charge-coupled device (CCD) camera and appropriate software.

The major technical challenge of array CGH is generating hybridization signals that are sufficiently intense and specific so that copy number changes can be detected. The signal intensity on an array element is affected by a number of factors including the base composition, the proportion of repetitive sequence content, and the amount of DNA in the array element available for hybridization.

Array elements made from genomic BAC clones typically provide more intense signals than elements employing shorter sequences such as cDNAs, PCR products, and oligonucleotides. The higher signals from the more complex array elements result in better measurement precision, allowing detection of single-copy transition boundaries—even in specimens with a high proportion of normal cells.

In another preferred embodiment, said hybridization-based assay is a fluorescence in situ hybridization (FISH) or FISH plus spectral karotype (SKY) (Liehr T. et al 2008 Fluorescence In Situ Hybridization (FISH)—Application Guide, Springer Berlin Heidelberg).

FISH allows to detect and localize the presence or absence of specific DNA sequences on chromosomes, for example, FISH allows localize the signal to a specific (tumor) cell type. FISH uses fluorescent probes that bind to only those parts of the chromosome with which they show a high degree of sequence similarity. Fluorescence microscopy can be used to find out where the fluorescent probe bound to the chromosomes.

The term “probe” as used herein refers to any ribopolynucleotide or desoxiribopolynucleotide sequence that specifically binds to only those parts of the chromosome with which they show a high degree of sequence similarity. The probe must be large enough to hybridize specifically with its target but not so large as to impede the hybridization process. There are many different FISH probes that can be used in the present invention; illustrative, non-limitative examples thereof include bacterial artificial chromosomes (BACs), Tiling Oligonucleotide Probes (TOPs), etc. The design of FISH probes is well know for a person skilled in the art (please see Bayani J, Squire J A. Curr Protoc Cell Biol. 2004 September; Chapter 22: Unit 22.4; Bayani J, Squire J. Curr Protoc Cell Biol. 2004 October; Chapter 22: Unit 22.5; Navin, N. et al. Bioinformatics, Volume 22, Number 19, 1 Oct. 2006, pp. 2437-2438(2)) Publisher: Oxford University Press). The probe can be tagged directly with fluorophores, with targets for antibodies, with biotin, etc. Tagging can be done in various ways, such as by nick translation, by PCR using tagged nucleotides, etc.

The sample can be fixed and in paraffin embedded, thus an additionally step of deparafination may be performed.

For hybridization, an interphase or metaphase chromosome preparation is produced. The chromosomes are firmly attached to a substrate, usually, a glass. Repetitive DNA sequences must be blocked by adding short fragments of DNA to the sample. The probe is then applied to the chromosome DNA and incubated for approximately 12 hours while hybridizing. Several wash steps remove all unhybridized or partially-hybridized probes. After standard post hybridization washes the slides are stained with the DNA staining probe such DAPI and mounted with a mounting agent such as antifade.

The results are then visualized and quantified by using, for example, a microscope that is capable of exciting the dye and recording images. If the fluorescent signal is weak, amplification of the signal may be necessary in order to exceed the detection threshold of the microscope. Fluorescent signal strength depends on many factors such as probe labeling efficiency, the type of probe, and the type of dye. Fluorescently-tagged antibodies or streptavidin are bound to the dye molecule. These secondary components are selected so that they have a strong signal. In a preferred embodiment, prior to imaging all slides are evaluated by a pathologist and regions of interest are identified based on histopathologic and quality criteria including, without excluding others, tumor content, appropriate fixation, necrosis and vascularity.

FISH experiments designed to detect or localize gene expression within cells and tissues rely on the use of a reporter gene, such as one expressing green fluorescent protein (GFP) and the like, to provide the fluorescence signal.

In an alternative technique to interphase or metaphase preparations, fiber FISH, interphase chromosomes are attached to a slide in such a way that they are stretched out in a straight line, rather than being tightly coiled, as in conventional FISH, or adopting a random conformation, as in interphase FISH. This is accomplished by applying mechanical shear along the length of the slide, either to cells that have been fixed to the slide and then lysed, or to a solution of purified DNA. The extended conformation of the chromosomes allows dramatically higher resolution—even down to a few kilobases (kb).

In a further particularly preferred embodiment, parallel to the detection of the polysomy/amplification of the EGFR locus and the LOH level of the PTEN gene, FISH control probes for chromosomes 10 and 7 are used. Those “FISH control probes” are probes that binds specific for the individual chromosomes, thus allowing the determination of the chromosome number. In a preferred embodiment, the FISH control probes are directed to alpha satellite sequences. Alpha satellite sequences, whilst highly repetitive, are specific to each individual chromosome. These sequences flank the centromeres and can present a target measured in megabases. In a preferred embodiment, these FISH control probes would be labeled with different colors than the EGFR and PTEN probes.

In a preferred embodiment, the PTEN FISH probe is a probe that hybridizes to the 10q23 region on chromosome 10 and contains sequences that flank both the 5′ and 3′ ends of the PTEN gene; in a more preferred embodiment the probe has between 300 and 400 kb. In a more preferred embodiment, the FISH probe for PTEN and the FISH control probe for chromosome 10 are the LSI PTEN (10q23)/CEP 10 dual color probe for PTEN (Vysis, Abbot Molecular) (Goberdhan, D., et al. Human Molecular Genetics 12 (2) (2003): 239-248; Eng, C., et al. Human Mutation 22 (2003): 183-198; Sasaki, H., et al. Am. J. of Pathology 159 (1) (2001): 359-367). Other probes are described by Cairns et al. (Cairns et al. 1997. Cancer Res 57; 4997-5000) and by Hermans et al. (Hermans et al. 2004 Genes Chrom Cancer, 39; 171-184). The LSI PTEN (10q23) is labeled with SpectrumOrange. The CEP 10 SpectrumGreen probe hybridizes to alpha satellite sequences specific to chromosome 10.

In a particular embodiment, the EGFR FISH probe is a probe that hybridizes to the 7p12 region of the chromosome 7 and contains the entire EGFR gene. In a more preferred embodiment, the FISH probe for the EGFR gene and the control probe for chromosome 10 are the LSI EGFR/CEP 7 dual color probe (Vysis, Abbot Molecular). In a particular embodiment the EGFR Probe is labeled with SpectrumOrange and covers an approximately 300 kb region of the 7p12 region of the chromosome 7 (Bredel, M., et al. 1999. Clin Cancer Res 5, 1786-92; Harris, A., et al. 1989. J Steroid Biochem 34, 123-31; Kitagawa, Y., et al. 1996. Clin Cancer Res 2, 909-14; Neal, D. E., et al. 1990. Cancer 65, 1619-25; Osaki, A., et al. 1992. Am J of Surg 164, 323-6; Pavelic, K., et al. 1993. Anticancer Res 13, 1133-7; Sauter, G., et al. 1996. Am J Pathol 148, 1047-53; Torregrosa, D., et al. 1997. Clin Chim Acta 262, 99-119). The CEP 7 probe, labeled with SpectrumGreen, hybridizes to the alpha satellite DNA located at the centromere of chromosome 7 (7p11.1-q11.1).

Other methods known in the arte may be used to determine copy number aberrations; illustrative, non-limitative examples thereof include oligonucleotide-based microarrays (Lucito, et al. 2003. Genome Res. 13:2291-2305; Bignell et al. 2004. Genome Res. 14:287-295; Zhao, et al. 2004. Cancer Research, 64(9):3060-71).

In another particular embodiment, the polysomy/amplification level of the EGFR gene and/or the LOH level of the PTEN gene is measured by using simple sequence length polymorphisms (or microsatelites) (Virmani A. K. et al. Genes chromosomes Cancer 1998, 21 (4) 308-319) or SNPs as genetic markers (Lindblad-toh K et al. Nat. Biotechnol. 2000, 18(9)1001-1005); in a particular embodiment, the polysomy/amplification level of the EGFR gene and/or the LOH level of the PTEN gene is measured by using a SNP array.

In another particular embodiment, the polysomy/amplification level of the EGFR gene and/or the LOH level of the PTEN gene is measured by polynucleotide sequencing, i.e., a method for determining the order of the nucleotide bases in a molecule of a polynucleotide. In a particular embodiment, the polynucleotide sequencing is performed by using a deep sequencing technology. In a more particular embodiment, the sequencing technology uses a large-scale parallel pyrosequencing system capable of sequencing roughly 400-600 megabases of DNA per run with 400-500 base pair read lengths on a suitable sequencer (e.g., Genome Sequencer FLX with GS FLX Titanium series reagents). The system relies on fixing nebulized and adapter-ligated DNA fragments to small DNA-capture beads in a water-in-oil emulsion. The DNA fixed to these beads is then amplified by PCR. Each DNA-bound bead is placed into a well on a PicoTiterPlate, a fiber optic chip. A mix of enzymes such as DNA polymerase, ATP sulfurylase, and luciferase are also packed into the well. The PicoTiterPlate is then placed into the GS FLX System for sequencing. This sequencing technology can sequence any double-stranded DNA and enables a variety of applications including de novo whole genome sequencing, re-sequencing of whole genomes and target DNA regions, metagenomics and RNA analysis. In another embodiment, the sequencing technology is the amplicon sequencing technology, i.e., an ultra deep sequencing designed to allow mutations to be detected at extremely low levels, and PCR amplify specific, targeted regions of DNA. This method is used to identify low frequency somatic mutations in cancer samples or discovery of rare variants in HIV infected individuals.

The determination of the level of the polysomy/amplification of EGFR and the level of LOH of PTEN, needs to be correlated with a standard reference value. Said standard reference values are generated by the person skilled in the art in form of a table that divide the patient in ascending number of copies of the EGFR gene or regarding to the level of LOH and ascending number of copies of the PTEN gene.

For EGFR, the reference values can be generated, without limitation, using for example, the classification of Capuzzo et al. (Cappuzzo et al. JNCI 2005; 4:643-55). In this classification, the patients are classified into six strata with ascending number of copies of the EGFR gene per cell according to the frequency of tumor cells with specific number of copies of the EGFR gene per chromosome 7. As it was mentioned before, control probes that detect the chromosome 7 for obtaining a ratio number of copies of EGFR gene/chromosome 7 are commercially available. For example, the CEP 7 SpectrumGreen probe (Vysis) that hybridizes to alpha satellite sequences specific to chromosome 7, can be used.

A non limitative example of a table for classifying a patient attending to the polysomy of the EGFR gene is:
1) disomy (D): ≤2 copies in >90% of the cells of the sample;
2) low trisomy (LT): ≤2 copies in ≥40% of the cells of the sample or 3 copies in 10%-40% of the cells of the sample or ≥4 copies in <10% of the cells of the sample;
3) high trisomy (HT): ≤2 copies in ≥40% of the cells of the sample or 3 copies in ≥40% of the cells of the sample or ≥4 copies in <10% of the cells of the sample;
4) low polysomy (LP): ≥4 copies in 10%-40% of the cells of the sample; and
5) high polysomy (HP): ≥4 copies in ≥40% of the cells of the sample or the presence of amplification (presence of tight EGFR gene clusters and a ratio of EGFR gene to chromosome of ≥2 or ≥15 copies of EGFR per cell in ≥10% of analyzed cells of the sample).

As a person skilled in the art will understand, the method [1] of the invention can be performed by using more than one sample of a patient. In such a case, it is considered that exist a “high polysomy level of the EGFR gene” when at least 50%, preferably more than 50%, more than 60%, more than 70%, more than 80%, more than 90%, more than 95%, or even more, of the samples of the patient are classified as “high polysomy” (HP) according to the classification criteria described before.

For the generation of a table for the classification of the patients attending to the level of the LOH and number of copies of the PTEN gene, the person skilled in the art could, for example, classify the patients into 4 strata with ascending number of copies of the PTEN gene per cell according to the frequency of tumor cells with specific number of copies of the PTEN gene per chromosome 10. As it was mentioned before, control probes that detect the chromosome 10 for obtaining a ratio number of copies of PTEN gene/chromosome 10 are commercially available. For example, the CEP 10 SpectrumGreen probe (Vysis) that hybridizes to alpha satellite sequences specific to chromosome 10, can be used.

A non limitative example of a table for classifying a patient attending to the polysomy/level of LOH of the PTEN gene is:
1) disomy (D): 2 copies of each probe in >90% of cells;
2) LOH: <2 copies of PTEN probe in >10% of cells (includes cromosome 10 monosomy or disomy with PTEN LOH, always in >10% cells);
3) polysomy (P): ≥3 copies of each probe (PTEN+CEP 10) in >10% of cells (it does not discriminate between high and low polisomy, or chromosome 10 trisomy); and
4) amplified (AMP): defined by a ratio of the PTEN gene to chromosome 10 of ≥2 per cell in ≥10% of analyzed cells.

As a person skilled in the art will understand, the method [1] of the invention can be performed by using more than one sample of a patient. In such a case, it is considered that exist a “high LOH level of the PTEN gene” when at least 50%, preferably more than 50%, more than 60%, more than 70%, more than 80%, more than 90%, more than 95%, or even more, of the samples of the patient are classified as “LOH” according to the classification criteria described before.

In the last step of the method [1] of the invention, a high LOH level of the PTEN gene with respect to said standard reference values and high expression levels and/or high polysomy/amplification of the EGFR gene are indicative of a good clinical outcome of the subject.

As a person skilled in the art understand, the samples can also be considered not appropriated for be included in any of the classification strata. Thus, in a preferred embodiment, additionally, samples sections of tissue from the patient are stained with colorants as hematoxylin and eosin (H&E) and reviewed by two or more persons skilled in the art (i.e., pathologists) to assess overall tumor content, necrosis and overall quality (e.g., thermal cautery effect, fixation with morphology, etc). In a preferred embodiment the samples used for determining the expression levels and/or the polysomy levels of the EGFR gene and the LOH level of the PTEN gene have at least 20%, at least 30%, at least 40%, at least 50%, at least 60%, at least 70%, at least 80%, at least 90%, at least 95% of tumor tissue in the sample, and have an appropriate fixation with acceptable morphology.

The teachings of the instant invention do not agree with the conclusions reached by Umesh et al. (Clinical Neuropathology. Vol. 28—No. 5/2009 (362-372)), who describe a method for predicting the patient outcome of glioma comprising the detection of EGFR amplification and PTEN LOH by immunohistochemistry (IHC) wherein it is stated that EGFR gene amplification associated with LOH of the PTEN gene is a trend to poor survival.

Effectively, as shown in the Example, the teachings of the present invention, which comprises combining EGFR polysomy/amplification (i.e., EGFR expression) with LOH of the PTEN gene wherein the LOH level of the PTEN gene is measured by PCR, a hybridization-based assay, sequencing or SNP arrays [the level of polysomy/amplification of the EGFR gene can be determined at the protein level or, alternatively, at the nucleic acid level] show that an EGFR amplification associated with LOH of the PTEN gene is a trend to good survival.

Assays performed by the inventors have shown that, apparently, the use of IHC to determine the level of the LOH of the PTEN gene is not suitable for accurately assess its relevant expression profile what is especially important in low expressing but not loss of PTEN protein tumors where such a critical difference will allow the true assessment of protein expression in this setting. Further, different results in an IHC assay can be attributed to specific antibodies and their sensitivity for assessing PTEN; there is documented literature to support the challenges for using IHC to assess PTEN when compared with FISH (e.g., Reid et al., British Journal of Cancer 2010, 102:678-684). DNA FISH methods are generally not impacted by the pre-analytic sample preparation, by contrast, such differences in fixation and specimen handling does impact on the PTEN antigen in tissue. One way around this is to use mathematical modeling with quantitative IF to assess true expression vs. Non-specific binding and ‘noise’ in the system.

Thus, the invention provides method for predicting in a more accurate way the clinical outcome of a subject suffering from glioma.

The findings of the inventors allow the determination of the clinical outcome of a patient suffering glioma measuring the LOH level of the PTEN gene. Thus, in a second aspect, the invention relates to a method for predicting the clinical outcome of a subject suffering from glioma, hereinafter referred to as the method [2] of the invention, said method comprising:
a) determining the LOH level of the PTEN gene in a sample from the subject, and
b) comparing said LOH level of the PTEN gene with a standard reference value,

wherein the LOH level of the PTEN gene is measured by PCR, or by a hybridization-based assay, or by sequencing, or by a SNP analysis; and

wherein a high LOH level of the PTEN gene with respect to said standard reference value, is indicative of a bad clinical outcome of the subject.

The term glioma has been previously defined. In a preferred embodiment, the glioma is a glioblastoma multiforme (GBM) and more preferably the glioblastoma is an early glioblastoma.

In a preferred embodiment, the clinical outcome is measured as survival.

The term “sample” has been previously defined in relation to the method [1] of the invention and can be applied to any type of biological sample from a patient, such as a biopsy sample, tissue, cell or fluid (whole blood, serum, saliva, semen, sputum, cerebral spinal fluid (CSF), urine, tears, mucus, sweat, milk, brain extracts and the like). In a particular embodiment, said sample is a tumour tissue sample or portion thereof. In a more particular embodiment, said tumor tissue sample is a brain tumor tissue sample from a patient suffering from glioma or a formalin embedded brain tissue sample.

The methods for determining the LOH level of the PTEN gene have been described above as well as the standard reference values used. In a preferred embodiment, the LOH level of the PTEN gene is determined by a hybridization-based assay, e.g., by FISH.

The invention also relates to a kit useful in the implementation of the methodology described herein. Thus, in another aspect, the invention relates to a kit comprising a set of agents capable of specifically determining the expression levels and/or the polysomy/amplification of EGFR and the LOH level of the PTEN gene and, optionally, a reagent for detecting a housekeeping gene or the protein encoded by said housekeeping gene and/or a reagent for detecting the chromosomes 7 and 10, wherein the set of agents capable of specifically determining the LOH level of the PTEN gene comprises a pair of oligonucleotide primers suitable for amplifying a specific fragment of the PTEN gene and/or an optionally labeled oligonucleotide probe which selectively binds to a target polynucleotide sequence on the chromosome region of the PTEN gene and/or reagents suitable for performing a sequencing reaction and/or reagents for performing an SNP analysis.

In a particular embodiment of the kit of the invention, the agents of the kit are capable of specifically detecting the mRNA levels of EGFR and/or PTEN genes or the levels of the EGFR and/or PTEN proteins, preferably, the levels of the EGFR protein.

Nucleic acids capable of specifically hybridizing with the EGFR and/or PTEN genes can be one or more pairs of primer oligonucleotides for the specific amplification of fragments of the mRNAs (or of their corresponding cDNAs) of said genes and/or one or more probes for the identification of one or more genes selected from said genes. Nucleic acids capable of specifically hybridizing with the EGFR and/or PTEN genes can be as well FISH probes.

Antibodies, or a fragment thereof, capable of detecting an antigen, capable of specifically binding to EGFR and/or PTEN proteins or to variants thereof are, for example, monoclonal and polyclonal antibodies, antibody fragments, Fv, Fab, Fab′ y F(ab′)2, ScFv, diabodies, triabodies, tetrabodies and humanised antibodies.

Said reagents, specifically, the probes and the antibodies, may be fixed onto a solid support, such as a membrane, a plastic or a glass, optionally treated in order to facilitate fixation of said probes or antibodies onto the support. Said solid support, which comprises, at least, a set of antibodies capable of specifically binding to EGFR and/or PTEN proteins or to variants thereof, and/or probes specifically hybridized with the EGFR and/or PTEN genes, may be used for the detection of the expression levels by means of the array technology.

The kits of the invention optionally comprise additional reagents for detecting a polypeptide encoded by a housekeeping gene or the mRNA encoded by said housekeeping genes. The availability of said additional reagent allows the normalization of measurements taken in different samples (e.g. the test sample and the control sample) to exclude that the differences in expression of the different biomarkers are due to a different amount of total protein in the sample rather than to real differences in relative expression levels. Housekeeping genes, as used herein, relates to genes which code for proteins which are constitutively expressed and carry out essential cellular functions. Preferred housekeeping genes for use in the present invention include β-2-microglobulin, ubiquitin, 18-S ribosomal protein, cyclophilin, GAPDH and actin.

In an embodiment, the kit of the invention may contain reagents suitable for performing a sequencing reaction and/or reagents for performing an SNP array, e.g., enzymes, nucleotides, etc.

In another embodiment, the invention relates to the use of a kit of the invention for predicting the clinical outcome of a subject suffering from glioblastoma multiforme, wherein if said agents detect a high expression level and/or high level of polysomy/amplification of the EGFR gene and a high LOH level of the PTEN gene, with respect to standard reference values, in a sample from said subject, then the clinical outcome of the subject is good.

Methods for detecting the expression levels of EGFR and PTEN and the methods for determining the polysomy of the EGFR gene and the LOH level of the PTEN gene as well as the standard reference values have been described previously (see, for example, method [1] of the invention).

In another embodiment, the invention relates to the use of the EGFR expression levels and/or polysomy/amplification levels of the EGFR gene and the LOH level of the PTEN gene as predictive marker of the clinical outcome of a glioma patient. In a particular embodiment, the glioma is a glioblastoma multiforme (GBM). In another particular embodiment, the clinical outcome is measure as survival.

In another particular embodiment, the invention relates to the use of erlotinib and/or temozolomide in the manufacture of a medicament for the treatment of a glioma in a subject suffering from a glioma, wherein the medicament is for a subject having a high LOH level of the PTEN gene, as measured by PCR, by a hybridization-based assay, by sequencing or by a SNP analysis, with respect to a standard reference value and high expression levels and/or high polysomy/amplification of the EGFR gene with respect to standard reference values. Alternatively, this inventive aspect can de defined as erlotinib and/or temozolomide for use in the treatment of a glioma in a subject suffering from a glioma, wherein the medicament is for a subject having a high LOH level of the PTEN gene, as measured by PCR, by a hybridization-based assay, by sequencing or by a SNP array, with respect to a standard reference value and high expression levels and/or high polysomy/amplification of the EGFR gene with respect to standard reference values. In a particular embodiment, the glioma is a glioblastoma multiforme (GBM). In another particular embodiment, the clinical outcome is measure as survival.

In another embodiment, the invention relates to the use of radiotherapy in a regime for the treatment of a glioma in a subject suffering from a glioma, wherein said subject has a high LOH level of the PTEN gene, as measured by PCR, by a hybridization-based assay, by sequencing or by a SNP analysis, with respect to a standard reference value and high expression levels and/or high polysomy/amplification of the EGFR gene with respect to standard reference values. In a particular embodiment, the glioma is a glioblastoma multiforme (GBM). In another particular embodiment, the clinical outcome is measure as survival.

### EXAMPLES

### I. Material and Methods

Patients and Specimens

Multiple formalin-fixed, paraffin-embedded blocks from fifty-six (56) primary brain tumor specimens were obtained from the Hospital Universitari de Belvitge. These patients were part of a longitudinal cohort and selected utilizing pre-determined inclusion criteria which included a primary diagnosis of any one of the following criteria: astrocytic, oligodendrocytic and glial tumor, with continuous follow-up for a median of 3 years and available tissue material. Clinical data was abstracted from the patient's clinical records utilizing a set of pre-determined clinical-pathologic and outcome data fields (see Table 1). The histologic breakdown of the tumor specimens were as follows:
I. Astrocytoma group, total 44 patients: pilocytic (1), diffuse (13), anaplastic (16), and glioblastoma multiforme (14);
II. Oligodendroglioma group, total 6 patients: oligodendroglioma (6); and
III. Mixed, total 4 patients: oligoastrocytoma (3), and anaplastic oligoastrocytoma (1).

A comprehensive review of the available clinical data files was performed on all patients in which formalin-fixed, paraffin-embedded (FFPE) samples/blocks were available for further analysis.

5 μm sections were obtained from all blocks, stained with hematoxylin and eosin (H&E) and reviewed by two pathologists to assess overall tumor content (≥50% tumor in at least one 200× field), necrosis and overall quality (e.g., thermal cautery effect, appropriate fixation with acceptable morphology, etc). Subsequent sections were then obtained for DNA FISH assays and quantitative immunofluorescence (IF) as outlined below. All blocks were retained for subsequent studies including RT-PCR.

TABLE 1
Clinical data fields for
entire cohort with breakdown
by diagnostic group

| | | | | Mixed/ | Non |
|---|---|---|---|---|---|
| | | Glioblastoma | Astrocytoma | others | available |
| | | N = 15 | N = 26 | N = 5 | N = 10 |
| Patients gender: | Male | 8 | 17 | 4 | 5 |
| | Female | 7 | 9 | 1 | 3 |
| | Non available | 0 | 0 | 0 | 2 |
| Patients race | caucasian | 15 | 25 | 5 | 8 |
| | Non available | 0 | 1 | 0 | 2 |
| Symptoms seizures | Yes | 4 | 13 | 2 | 5 |
| | No | 11 | 13 | 2 | 3 |
| | Non available | 0 | 0 | 1 | 2 |
| Symptoms high intracranial | Yes | 7 | 5 | 1 | 1 |
| pressure | No | 8 | 21 | 3 | 7 |
| | Non available | 0 | 0 | 1 | 2 |
| Number of lesions in | Image 1 | 12 | 21 | 5 | 7 |
| pretreatment | Image 2 | 1 | 1 | 0 | 0 |
| | multiples | 2 | 3 | 0 | 1 |
| | Non available | 0 | 1 | 0 | 2 |
| 1 location of lesions in | Frontal lobe | 10 | 10 | 3 | 5 |
| pretreatment image: | Temporal lobe | 3 | 3 | 0 | 1 |
| | Parietal lobe | 1 | 6 | 1 | 0 |
| | Operculum | 0 | 1 | 0 | 0 |
| | Brain stem | 1 | 2 | 1 | 0 |
| | Spinal cord | 0 | 1 | 0 | 0 |
| | Optic nerve | 0 | 0 | 0 | 1 |
| | Inter- | 0 | 1 | 0 | 0 |
| | hemispheric | | | | |
| | cisure | | | | |
| | Cerebellum | 0 | 0 | 0 | 1 |
| | | 0 | 1 | 0 | 0 |
| | Non available | 0 | 1 | 0 | 2 |
| 2 location of lesions in | Temporal lobe | 1 | 1 | 0 | 1 |
| pretreatment image | Parietal lobe | 3 | 1 | 0 | 0 |
| | Occipital lobe | 2 | 1 | 0 | 1 |
| | Corpus | 0 | 1 | 0 | 0 |
| | callosum | | | | |
| | Basal ganglia | 2 | 0 | 0 | 0 |
| | Brain stem | 0 | 1 | 0 | 0 |
| | Thalamus | 0 | 0 | 0 | 1 |
| | Non available | 7 | 21 | 5 | 7 |
| 1 side of lesions in pretreatment | Right | 9 | 12 | 2 | 6 |
| image | Left | 4 | 11 | 3 | 2 |
| | Unknown | 0 | 1 | 0 | 0 |
| | Non available | 2 | 2 | 0 | 2 |
| 2 side of lesions in pretreatment | Left | 1 | 1 | 0 | 1 |
| image | Non available | 14 | 25 | 5 | 9 |
| Type of specimen | Biopsy | 1 | 6 | 0 | 4 |
| | Stereotaxic | 2 | 4 | 0 | 1 |
| | biopsy | | | | |
| | Resection | 12 | 16 | 5 | 3 |
| | Non available | 0 | 0 | 0 | 2 |
| Type of postsurgical removal | Complete | 4 | 11 | 1 | 6 |
| | Incomplete | 10 | 12 | 4 | 2 |
| | Non available | 1 | 3 | 0 | 2 |
| Radiation therapy | Yes | 7 | 12 | 3 | 1 |
| | No | 8 | 12 | 2 | 7 |
| | Non available | 0 | 2 | 0 | 2 |
| Type of radiation | Focal | 5 | 8 | 0 | 1 |
| | Non available | 10 | 18 | 5 | 9 |
| Total dose of radiation (**) | | 60.0 (60.0-60.0) | 60.0 (40.0-60.0) | 51.0 | 60.0 |
| (Available data: n = 20) | | | | (48.0-54.0) | (60.0-60.0) |
| Chemotherapy | Yes | 2 | 9 | 1 | 1 |
| | No | 13 | 15 | 4 | 7 |
| | Non available | 0 | 2 | 0 | 2 |
| Type of first chemotherapy | BCNU | 0 | 7 | 0 | 0 |
| | TMZ | 2 | 1 | 0 | 0 |
| | PCV | 0 | 1 | 0 | 1 |
| | Non available | 13 | 17 | 5 | 9 |
| Recurrence or progression | Yes | 4 | 13 | 4 | 6 |
| | No | 9 | 10 | 1 | 1 |
| | Non available | 2 | 3 | 0 | 3 |
| Type of first recurrence | Focal | 3 | 8 | 4 | 6 |
| | Diffuse | 1 | 1 | 0 | 0 |
| | Multiple | 0 | 2 | 0 | 0 |
| | Non available | 11 | 15 | 1 | 4 |
| Treatment of first recurrence | Yes | 3 | 10 | 4 | 5 |
| | No | 1 | 1 | 0 | 1 |
| | Non available | 11 | 15 | 1 | 4 |
| 1 type of treatment of first | Radiation | 0 | 2 | 1 | 1 |
| recurrence | Chemotherapy | 2 | 3 | 1 | 0 |
| | Surgery | 1 | 4 | 2 | 4 |
| | Radiosurgery | 0 | 1 | 0 | 0 |
| | Non available | 12 | 16 | 1 | 5 |
| 2 type of treatment of first | Radiation | 0 | 2 | 0 | 4 |
| recurrence | Chemotherapy | 0 | 1 | 0 | 0 |
| | Non available | 15 | 23 | 5 | 6 |
| 3 type of treatment of first | Chemotherapy | 0 | 1 | 0 | 1 |
| recurrence | Non available | 15 | 25 | 5 | 9 |
| Karnovsky value pretreatment | | 70.0 (40.0-100) | 90.0 (20.0-100) | 95.0 | 100 (60.0-100) |
| (**) (Available data: n = 49) | | | | (90.0-100) | |
| Karnovsky value postreatment | | 60.0 (30.0-90.0) | 80.0 (10.0-100) | 95.0 | 100 (30.0-100) |
| (**) (Available data: n = 46) | | | | (80.0-100) | |
| Death of patient | Yes | 15 | 15 | 1 | 1 |
| | No | 0 | 7 | 3 | 5 |
| | Non available | 0 | 4 | 1 | 4 |

DNA EGFR and PTEN FISH

Using the LSI EGFR/CEP 7 dual color probe (Vysis, Abbot Molecular) for EGFR and the LSI PTEN (10q23)/CEP 10 dual color probe for PTEN (Vysis, Abbot Molecular), DNA FISH including hybridization, washing and fluorescence detection was performed on all specimens in the cohort as per standard protocols (Smith et al., 2001. JNCI; 93:1246-1256). Briefly, paraffin sections were dewaxed in xylenes, microwaved in 10 mmol/L sodium citrate (pH 6.0) solution for 5-10 minutes, cooled to room temperature, rinsed, and then treated with pepsin HCl for 5 minutes at 37° C. before being rinsed and dehydrated. The prewarmed probe mixture was applied to the slides, and a cover-slip sealed in place with rubber cement. The slides were then denatured at 85° C. for 4 to 6 minutes using an automated hybridization chamber and then incubated overnight at 37° C. After standard post-hybridization washes the slides were stained with the DNA stain DAPI and mounted with antifade (Vectashield). Prior to imaging all H&E slides were evaluated by a pathologist and regions of interest were identified based on histopathologic and quality criteria including tumor content, appropriate fixation, necrosis and vascularity. A H&E image of individual GBM cases is illustrated in FIG. 5A. All slides were imaged using a Nikon immunofluorescent microscope by a trained scientist blinded to outcome. Criteria for FISH anomalies were defined by use of histologically normal brain specimens. Simple gain required 10% or more of nuclei with three or more locus-specific probe signals. Loss of the q arm of chromosome 10 required the overall mean PTEN/CEP10 ratio to be less than 0.90. Amplification was applied for both EGFR and PTEN and required that the ratio must be 2 or more and that 10% or more of nuclei had more than three EGFR and or PTEN signals. Representative regions of interest were acquired for documentation of signal in all specimens (Examples of representative images of FISH AMP/HP of EGFR and monosomy of PTEN are shown in FIGS. 5B and C, respectively). For EGFR FISH we followed the Capuzzo et al. (Cappuzzo F, et al., JNCI 2005; 4: 643-55) classification scheme and identified 6 categories for EGFR status in all tumor specimens. These individual states included: D=disomy, HT=high trisomy, LT=low trisomy, HP=high polysomy, LP=low polysomy, and AMP=amplification. In this schema both HP and AMP together are considered amplification. PTEN status was characterized as D=disomy, LOH=>10%, P=polysomy, AMP=amplified and NE=not evaluable. All cases were reviewed by a single observer and recorded anonymously with respect to tumor type or outcome.

Quantitative Immunofluorescence (IF)

Utilizing previous established methods (Cordon Cardo et al., 2007 JCI 2007; 117; 1876-83; Donovan et al., 2008 JCO; 26:3923-3929) the multiplex-1 (mplex1) assay was constructed incorporating antibodies to: GFAP (glial fibrillary acidic protein), PTEN (phosphatase and tensin homolog), pAKT and Ki67 combined with the nuclear stain DAPI (4′,6-diamidino-2-phenylindole)—see Table 2 for a complete review of antibodies used within the mplex1 assay. The individual antibodies were evaluated in simplex IF assays using control tissues and cell lines to confirm expression sensitivity and specificity prior to inclusion in the multiplex-1 assay.

TABLE 2
Reagent list

| Antibody | Vendor | Catalog # | Dilution | Isotype | Labels | Samples |
|---|---|---|---|---|---|---|
| PTEN | Neomarkers | | 1:30 | M IgG1 | 2X M 488 | GBM cases, |
| | | | | | | NL brain LNCaP |
| | | | | | | Tonsil |
| Ki67 | Dako | | 1:100 | M IgG1 | 2X M 555 | Same |
| pAKT | CST | #3787 | 1:25 | R IgG | 2X R 594 | Same |
| GFAP | Dako | | 1:200 | M IgG1 | 2X M 647 | Same |

In brief, FFPE samples were de-paraffinized, rehydrated and subjected to an antigen retrieval process with the Reveal buffer system (Biocare Medical). A series of four antibodies were combined with DAPI into a multiplex immunofluorescent assay. The reagents listed in Table 2 were differentially labeled and with the identified fluorochromea and then sequentially applied onto individual sections using a Nemesis 7200 immunostainer (BioCare Medical). A minimum of three images or regions of interest (ROI) were acquired by a pathologist (blinded to outcome) using a Nikon 90i immunofluorescent microscope equipped with a CRI spectral imaging system (CRI). Utilizing pre-developed unmixing libraries and CRI software, individual gray scale images were generated which represent the antibody—filter combination under investigation. The individual images were evaluated for signal:noise ratio, cellular distribution and co-distribution with other antibody-fluorochrome labeled reagents. Using the existing CRI software manual thresholds were created for individual antibody-fluorochrome combinations to maximize signal:noise and preserving distribution. Quantitative metrics were generated using the software to identify percentages of individual cell populations exhibiting a positive signal (based on the threshold), normalized to the tumor region under evaluation. A series of prostate cancer cell lines including LNCaP, PC3 and DU145 (ATCC) were obtained, grown to confluency, harvested as agar cell pellets, fixed and embedded in paraffin and then all three cell lines were placed into a cell array for quality control both during assay development and investigation of brain tumor cases.

Results and Discussion

Patients and Specimens

There were 54 patients with available clinical records and FFPE tissue samples appropriate for evaluation. The breakdown of patients is listed in Table 3 with the majority of patients in the astrocytoma diagnostic category. The individual demographic features including sex, location of mass, treatment and outcome data are outlined in detail in Table 1.

TABLE 3
Diagnosis category of the patients

| | Pure | | Oligo- | Mixed/ |
|---|---|---|---|---|
| Original codes | GBM | Astrocytoma | dendroglioma | other |
| 001: astrocytoma grade II | 0 | 10 | 0 | 0 |
| 002: astrocytoma grade | 0 | 16 | 0 | 0 |
| III | | | | |
| 003: glioblastoma | 15 | 0 | 0 | 0 |
| 004: oligoastrocytoma | 0 | 0 | 0 | 3 |
| grade II | | | | |
| 005: oligoastrocytoma | 0 | 0 | 0 | 2 |
| grade III | | | | |
| 006: oligodendroglioma | 0 | 0 | 4 | 0 |
| grade II | | | | |
| 007: oligodendroglioma | 0 | 0 | 1 | 0 |
| grade III | | | | |
| 008: pilocytic | 0 | 2 | 0 | 0 |
| astrocitoma | | | | |
| 009: gliomatosis cerebri | 0 | 0 | 0 | 1 |
| 999: Non available | 0 | 0 | 0 | 2 |

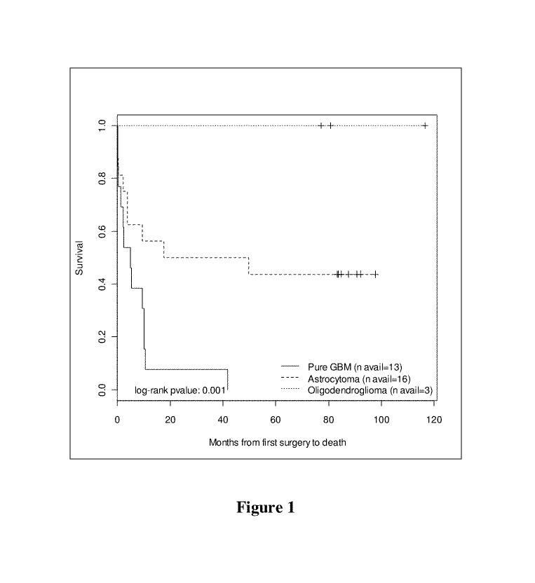

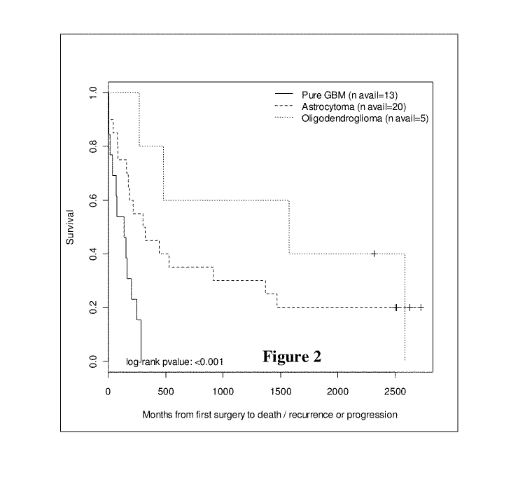

In order to understand general survival trends within the cohort and in particular the glioblastoma group vs. others we performed Kaplan-Meier survival function analyses to estimate survival between the three groups. FIG. 1 is the Kaplan-Meier survival curve demonstrating reduced survival in the glioblastoma group vs. the anaplastic astrocytoma and mixed categories (log-rank test P=0.001). FIG. 2 is a second Kaplan-Meier survival function curve which evaluates a more composite end-point which includes progression free and overall survival in the same three diagnostic categories (log-rank test P<0.001). In agreement with the literature, the glioblastoma group has a reduced overall and progression free survival when compared to the astrocytoma and pure oligodendroglioma group which has the longest survival time of all three diagnostic categories. As evident from the curves, a subset of the anaplastic astrocytoma group appears to behave like the glioblastoma category.

EGFR and PTEN FISH

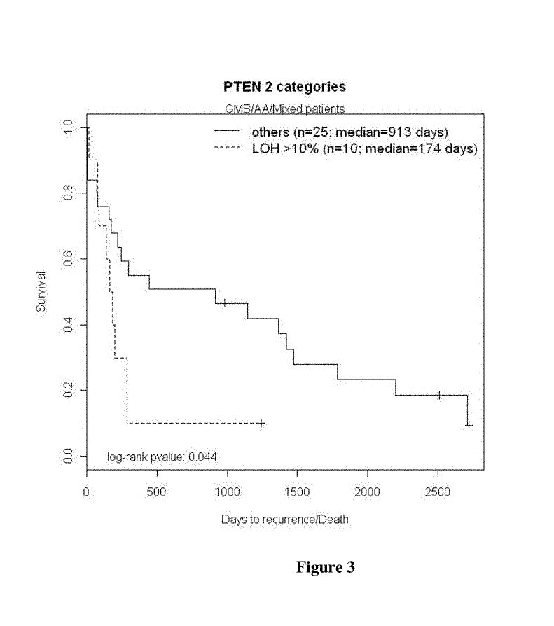

Given the frequency of EGFR amplification (approximately 40%) and reported role that EGFR plays in the development of glioblastoma, the first DNA FISH study performed was on the characterization of EGFR. Using the scoring methods of both Cappuzzo et al. (Capuzzo et al., 2005 JNCI; 4:643-55) and Smith et al. (Smith et al., 2001 JNCI; 93:1246-1256), we investigated EGFR profiles in all brain tumor cases in the group. Given the overall cohort size we opted to further classify patients as positive if they exhibited either polysomy and/or amplification of the EGFR locus or chromosome 7. This is in contrast to some reported studies which independently characterize EGFR DNA FISH as either polysomy or amplification. In our study 71% of the GBM patients had EGFR polysomy/amplification compared with 45% in the anaplastic astrocytoma group. We did not find EGFR amplification or polysomy to be associated with survival when examining the entire cohort or individual subgroups (all patients log-rank test P=0.8; glioblastoma only patients P=0.1). Dual-color FISH was also performed with probes for PTEN utilizing a similar scoring system as EGFR; however in addition to disomy and polysomy we included the loss of PTEN as higher than 10% in tumor epithelial nuclei. In our studies 61% of GBM samples had PTEN LOH compared with 25% in the anaplastic astrocytoma group. There is sparse literature on the evaluation of PTEN by DNA FISH in brain tumor samples with most studies using more molecular techniques such as sequencing, RT-PCR and immunohistochemistry (IHC). Noteworthy in our cohort, PTEN LOH was univariately and statistically associated with reduced survival in the combined glioblastoma multiforme, astrocytoma and mixed patient group (n=25 without LOH, median survival 913 D vs. n=10 with LOH, median survival 174 D; log-rank test p=0.04) [see FIG. 3].

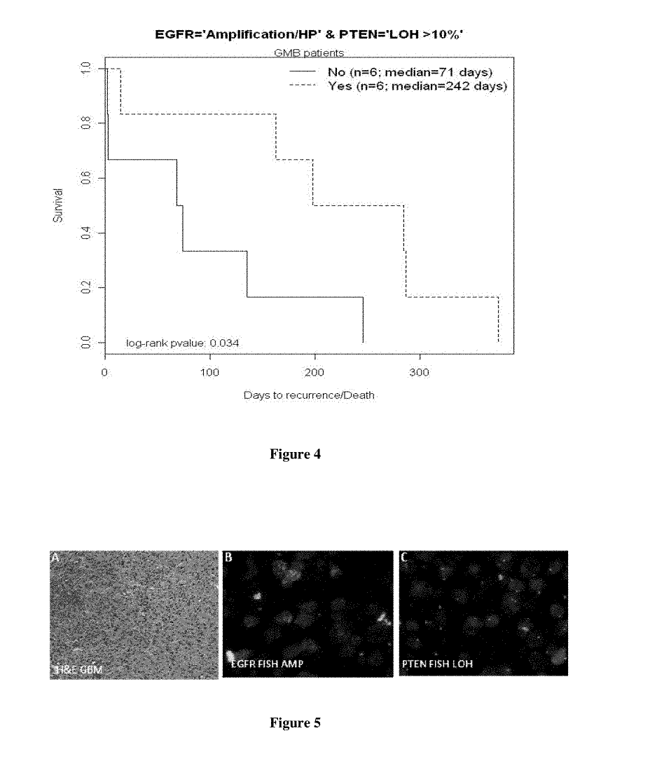

Neither EGFR polysomy/amplification or PTEN loss were significant, independent predictors for survival in the glioblastoma only group (P=0.1); however, when combined we observed a trend towards significance with an increase in overall survival (n=6 with both EGFR/PTEN, median survival 242 D vs. n=6 without, median survival 71 D; log-rank test P=0.034) (FIG. 4). The hypothesis is that the combination of PTEN LOH and EGFR amplification in glioblastoma may represent a subset of patients with a tumor phenotype more amenable to radiation and/or temozolomide treatment.

TABLE 4
Clinical characteristics of patients with previous filter conditions.

| Idalthia | gender | age | sympseiz | sympticp | imagelesions | imagelocat_1 | imagelocat_2 | imageside_1 | imageside_2 | diagn |
|---|---|---|---|---|---|---|---|---|---|---|
| L08-00080 | female | 50 | no | yes | 1 | frontal lobe | NA | left | NA | glioblastoma |
| L08-00084 | female | 65 | no | yes | 1 | frontal lobe | NA | right | NA | glioblastoma |
| L08-00085 | male | 71 | no | yes | 1 | temporal lobe | NA | right | NA | glioblastoma |
| L08-00087 | male | 66 | no | no | 1 | frontal lobe | parietal lobe | right | NA | glioblastoma |
| L08-00090 | female | 74 | no | no | 1 | parietal lobe | occipital lobe | right | NA | glioblastoma |
| L08-00091 | male | 68 | no | no | 1 | temporal lobe | NA | right | NA | glioblastoma |
| L08-00094 | female | 66 | yes | no | 1 | frontal lobe | temporal lobe | right | NA | glioblastoma |
| L08-00097 | male | 50 | yes | yes | 1 | temporal lobe | NA | right | NA | glioblastoma |
| L08-00098 | female | 49 | yes | no | 1 | frontal lobe | parietal lobe | left | NA | glioblastoma |
| L08-00106 | male | 68 | yes | yes | multiples | frontal lobe | basal ganglia | NA | NA | glioblastoma |
| L08-00109 | female | 60 | no | no | 2 | frontal lobe | occipital lobe | left | NA | glioblastoma |
| L08-00128 | male | 37 | no | yes | 1 | frontal lobe | NA | right | left | glioblastoma |

| Idalthia | diagnspec | psiremoval | recur.progr | recurrtype | death |
|---|---|---|---|---|---|
| L08-00080 | resection | complete | yes | NA | yes |
| L08-00084 | resection | complete | yes | focal | yes |
| L08-00085 | resection | complete | yes | focal | yes |
| L08-00087 | resection | incomplete | no | NA | yes |
| L08-00090 | biopsy | incomplete | no | NA | yes |
| L08-00091 | resection | complete | no | NA | yes |
| L08-00094 | resection | NA | no | NA | yes |
| L08-00097 | resection | incomplete | no | NA | yes |
| L08-00098 | resection | incomplete | yes | focal | yes |
| L08-00106 | stereothaxic | incomplete | no | NA | yes |
| | biopsy | | | | |
| L08-00109 | stereothaxic | incomplete | no | NA | yes |
| | biopsy | | | | |
| L08-00128 | resection | incomplete | yes | diffuse | yes |

NA: data non-available
Variable Info Table 4:
gender = “patients gender”
age = “patients age”
sympseiz = “symptoms seizures”
sympticp = “symptoms high intracranial pressure”
imagelesions = “number of lesions in pretreatment image”
imagelocat_1 = “location of 1st lesion in pretreatment image”
imagelocat_2 = “location of 2nd. lesion in pretreatment image”
imageside_1 = “side of 1st. lesion in pretreatment image”
imageside_2 = “side of 2nd. lesion in pretreatment image”
diagn = “pathological diagnosis”
diagnspec = “type of specimen”
psiremoval = “type of postsurgical removal”
recur.progr = “recurrence or progression”
recurrtype = “type of first recurrence”
death = “death of patient”

In Table 4, 12 patients were included, 6 with the EGFR AMP/PTEN LOH phenotype and 6 without (see column 2), EGFR/PTEN status (yes/no with respect to phenotype). Of the 6 Glioblastoma multiforme (GBM) patients with this phenotype, 3 exhibited no evidence of clinical recurrence. The 3 patients who did recur in the EGFR AMP/PTEN LOH positive group had received radiation and one received both radiation and the chemotherapeutic agent temozolomide (TMZ). Although the number of patients is small the data suggest that GBM patients with a tumor exhibiting the EGFR AMP/PTEN LOH have a better overall clinical outcome than patients without this phenotype regardless of current treatment including radiation and chemotherapy. We conclude that the identified phenotype which involves both EGFR signaling and PTEN loss may reflect an overall good acting GBM, irrespective of current treatment modalities and that newer investigative therapies and clinical trials would benefit from focusing on the intersect of these two pathways to further improve outcome.

1-20. (canceled)

21. A method for predicting the clinical outcome of and treating a subject suffering from glioblastoma multiforme (GBM) that comprises:
a) obtaining a sample from the same subject,
b) determining the expression level or the polysomy/amplification level of the EGFR gene and the LOH level of the PTEN gene in a sample from the subject, wherein the LOH level of the PTEN gene is measured by PCR, by a hybridization-based assay, by sequencing technology, or by a SNP analysis,
c) comparing said expression level or the polysomy/amplification level of the EGFR gene and the LOH level of the PTEN gene with standard reference values, wherein a high LOH level of the PTEN gene with respect to said standard reference value and a high expression level and/or high level of polysomy/amplification of the EGFR gene with respect to said standard reference values are indicative of a good clinical outcome of the subject, and
d) administering an effective amount of erlotinib and/or temozolomide to the subject, or administering a regime in combination with radiotherapy to the subject.

22. The method according to claim 1, wherein the clinical outcome is measured as survival.

23. The method according to claim 1, wherein the sample is a tumor tissue sample.

24. The method according to claim 1, wherein the expression level of the EGFR gene is measured by determining the mRNA and/or protein expression level of said gene.

25. The method according to claim 1, wherein said hybridization-based assay comprises a Southern blot, in situ hybridization (ISH), fluorescence in situ hybridization (FISH), or a comparative genomic hybridization (CGH) assay.

26. The method according to claim 1, wherein the LOH level of the PTEN gene is determined by FISH.

27. The method according to claim 1, wherein the glioblastoma is early glioblastoma.

28. A method for predicting the clinical outcome of and treating a subject suffering from glioblastoma multiforme (GBM) that comprises:
a) obtaining a sample from the same subject,
b) determining the LOH level of the PTEN gene in a sample from the subject, wherein the LOH level of the PTEN gene is measured by PCR, or by a hybridization-based assay, or by sequencing, or by a SNP analysis,
c) comparing said LOH level of the PTEN gene with a standard reference value,
wherein the LOH level of the PTEN gene is measured by PCR, or by a hybridization-based assay, or by sequencing, or by a SNP analysis, wherein a high LOH level of the PTEN gene with respect to said standard reference value, is indicative of a bad clinical outcome of the subject; and
d) administering an effective amount of erlotinib and/or temozolomide to the subject, or administering a regime in combination with radiotherapy to the subject.

29. The method according to claim 8, wherein the clinical outcome is measured as survival.

30. The method according to claim 8, wherein the sample is a tumor tissue sample.

31. The method according to claim 8, wherein said hybridization-based assay comprises a Southern blot, in situ hybridization (ISH), fluorescence in situ hybridization (FISH), or a comparative genomic hybridization (CGH) assay.

32. The method according to claim 8, wherein the LOH level of the PTEN gene is determined by FISH.

33. The method according to claim 8, wherein the glioblastoma is early glioblastoma.

34. The method for the treatment of a glioma in a subject suffering from a glioma comprising the administration to said subject of erlotinib and/or temozolomide wherein said subject has high LOH levels of the PTEN gene, as measured by PCR by a hybridization-based assay, or by sequencing or by a SNP analysis, with respect to a standard reference value and high expression levels and/or high polysomy/amplification of the EGFR gene with respect to standard reference values.

35. The method for the treatment of glioma in a subject suffering from a glioma comprising administering to said subject a regime in combination with radiotherapy wherein said subject has a high LOH level of the PTEN gene, as measured by PCR, by a hybridization-based assay, by sequencing or by a SNP analysis, with respect to a standard reference value and high expression levels and/or high polysomy/amplification of the EGFR gene with respect to standard reference values.


SEQUENCE LISTING (13 sequences)

SEQ ID NO: 1 - DNA, 5572 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcaaaaaaaaaaaaaaaaaaaaaaaaaaa

SEQ ID NO: 2 - protein, 403 aa, Homo sapiens [sequence omitted]

SEQ ID NO: 3 - DNA, 8229 bp, Mus musculus
gcggcaggatacgcgcttgggcgtcgggacgcggctgcgctcagctctctcctctcggaagctgcagccatgatggaagtttgagagttgagccgctgtgaggccaggcccggcgcaggcgagggagatgagagacggcggcggccacggcccagagcccctctcagcgcctgtgagcagccgcgggggcagcgccctcggggagccggccgggcggcggcggcggcagcggcggcgggcctcgcctcctcgtcgtctgttctaaccgggcagcttctgagcagcttcggagagagacggtggaagaagccgtgggctcgagcgggagccggcgcaggctcggcggctgcacctcccgctcctggagcgggggggagaagcggcggcggcggccgcggctccggggagggggtcggagtcgcctgtcaccattgccagggctgggaacgccggagagttgctctctccccttctcctgcctccaacacggcggcggcggcggcggcacgtccagggacccgggccggtgttaagcctcccgtccgccgccgccgcaccccccctggcccgggctccggaggccgccggaggaggcagccgctgcgaggattatccgtcttctccccattccgctgcctcggctgccaggcctctggctgctgaggagaagcaggcccagtctctgcaaccatccagcagccgccgcagcagccattacccggctgcggtccagggccaagcggcagcagagcgaggggcatcagcgaccgccaagtccagagccatttccatcctgcagaagaagcctcgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaatattattgctatgggatttcctgcagaaagacttgaaggtgtatacaggaacaatattgatgatgtagtaaggtttttggattcaaagcataaaaaccattacaagatatacaatctatgtgctgagagacattatgacaccgccaaatttaactgcagagttgcacagtatccttttgaagaccataacccaccacagctagaacttatcaaacccttctgtgaagatcttgaccaatggctaagtgaagatgacaatcatgttgcagcaattcactgtaaagctggaaagggacggactggtgtaatgatttgtgcatatttattgcatcggggcaaatttttaaaggcacaagaggccctagatttttatggggaagtaaggaccagagacaaaaagggagtcacaattcccagtcagaggcgctatgtatattattatagctacctgctaaaaaatcacctggattacagacccgtggcactgctgtttcacaagatgatgtttgaaactattccaatgttcagtggcggaacttgcaatcctcagtttgtggtctgccagctaaaggtgaagatatattcctccaattcaggacccacgcggcgggaggacaagttcatgtactttgagttccctcagccattgcctgtgtgtggtgatatcaaagtagagttcttccacaaacagaacaagatgctcaaaaaggacaaaatgtttcacttttgggtaaatacgttcttcataccaggaccagaggaaacctcagaaaaagtggaaaatggaagtctttgtgatcaggaaatcgatagcatttgcagtatagagcgtgcagataatgacaaggagtatcttgtactcaccctaacaaaaaacgatcttgacaaagcaaacaaagacaaggccaaccgatacttctctccaaattttaaggtgaaactatactttacaaaaacagtagaggagccatcaaatccagaggctagcagttcaacttctgtgactccagatgttagtgacaatgaacctgatcattatagatattctgacaccactgactctgatccagagaatgaaccttttgatgaagatcagcattcacaaattacaaaagtctgattttttttttcttatcaagagggataaaataccatgaaaaaaaaaaaacttgaataaactgaaatggaccttttttttttttttttttttttaaatggcaataggacattgtgtcagattgcagttataggaacaattctcttctcctgaccaatcttgttttaccctatacatccacagggttttgacacttgttgtccagttaaaaaaaggttgtgtagctgtgtcatgtatatacctttttgtgtcaaaaggacatttaaaattcaattaggataaataaaagatggcactttcccattttattccagttttataaaaagtggagacaggctgatgtgtatacgcaggagtttttcctttattttctgtcaccagctgaagtggctgaagagctctgattcccgggttcacgtcctacccctttgcacttgtggcaacagataagtttgcagttggctaaggaagtttctgcagggttttgttagattctaatgcatgcacttgggttgggaatggagggaatgctcagaaaggaatgtttctacctgggctctggaccatacaccatctccagctccttagatgcacctttctttagcatgctccacttactaatctggacatccgagagattggctgctgtcctgctgtttgtttgtgcattttaaagagcatattggtgctagacaaggcagctagagtgagtatatttgtagtggggtacaggaatgaaccatctacagcatcttaagaatccacaaaggaagggatataaaaaaagtggtcatagatagataaaagacacagcagcaatgacttaaccatacaaatgtggaggctttcaacaaaggatgggctggaaacagaaaatttgacaatgatttattcagtatgctttctcagttgtaatgactgctccatctcctatgtaatcaaggccagtgctaagagtcagatgctattagtccctacatcagtcaacaccttacctttatttttattaattttcaatcatatacctactgtggatgcttcatgtgctggctgccagtttgtttttctccttaaatattttataattcttcacaggaaatttcaacttgagattcaacagtaagcaggttttgttttttttttttcctagagattgatgatgcgcgtcctcagtccagtggctgtcagacgttcagcccctttgaccttacacattctattacaatgagttttgcagttttgcacattttttttaaatgtcattaactgttagggaattttacttgaatactgaatacatataatgtgtatattaaaaaagtcattgtttgtgttaaaaaagaaattagagttgcagtaaatttacagcactgcacgaataataaggcattgaagtttttcagtagaaattgtcctacagatgctttatcgacttgctattggaagaatagatcttcttaaatgtgcagtgttgagtcacttcgttatagtggtagagttgggattagggcttcaattttacttcttaaatatcattctatgtttgatatgcccagactgcatacaatttaaagcaagagtacaactactatcgtaatggtaatgtgaagatgctattacaaaggatctcctcccaacccctcgggaatttggtgtctttcaaattatatcttgaccttgacatttgaatatccagccattattagatttcttaatggtgtgaagtcccattttcaataacttattggtgctgaaattgttcactagctgtggtctgacctagttaatttacaagtacagattgcataggacccactagagaagcatttatagtttgatggtaagtagattaggcagaacgccatctaaaatattcttagaaaataatgttgatgtattttccatacctcatcagtttcactcaaccaataaagtttttaaaattgtaacaaagctcttaggatttacacatttatatttaaacattgatacatgaatattgactgactgttgataaagtcagagacaacttttcctgagatctcaccatggaaatctgtacacccccttgtctttcctaaaagctgaaagtggctgactaaaatgcaaagcagctgttgatgttttgaagatagtgataaacactgttctttgttagttttgggcacagcatgctaaactataacttgtattgttccaatatgtaacacagagggccaggtcatgaataatgacattacaatgggctgttgcactgttaatatttttcctttggaatgtgaaggtctgaatgagggttttgattttgaatgtttcagtgtttttgagaagccttgcttacattttatggtgtagtcattggaaatggaaaaatggcattatatatatattatatatatataaatatatatattatacatactctccttactttatttcagttaccatccccatagaatttgacaagaattgctatgactgaaagggttttgagtcctaattcaaactttctttatgacagtattcacgattagcctgaagtgcattctgtaggtgatctctcccgtgtttctggaatgctttcttagactcttggatgtgcagcagcttatgtgtctgaaatgacttgaaggcatcacctttaagaaggcttacagttgggccccgtacatcccaagtcctctgtaattcctcttggacatttttgccataattgtaaaagggtagttgaattaaatagcgtcaccattctttgctgtggcacaggttataaacttaagtggagtttaccggcagcatcaaatgtttcagctttaaaaataaaagtaggttacaagttacatgtttagttttagaaaatttgtgcaatatgttcataacgatggctgtggttgccacaaagtgcctcgtttacctttaaatactgttaatgtgtcgtgcatgcagacggaaggggtggatctgtgcactaaacggggggcttttactctagtattcggcagagttgccttctacctgccagctcaaaagttcgatctgttttcatatagaatatatatactaaaaccatccagtctgtaaaacagccttaccccgattcagcctcttcagatactcttgtgctgtgcagcagtggctctgtgtgtaaatgctatgcactgaggatacacaaatatgacgtgtacaggataatgcctcataccaatcagatgtccatttgttactgtgtttgttaacaaccctttatctcttagtgttataaactccacttaaaactgattaaagtctcattcttgtcattgtgtgggtgttttattaaatgagagtatttataattcaaattgcttaaatccattaaaatgttcagtaatgggcagccacatatgattacaaagttcctgtgcatttttctatttttccccctccttgctatccttccaagcaaagcatctttctgtcatcttggtagacacatacctgtctactcatggttaagaagagcactttaagccttagtcatcacttaataagttattccaggcacagtaaaaagttcaaggttcttggaaaacggtgcttatttctcttcttataagccagatgtctgaagatagccctaaccccaagaacgggcttgatgtctcaggtctgttctgtggctttctgttttttttaacactgcagttggccatcagcacatgggaggtttcatcgggacttgtccagagtagtaggctcaaatatactatctcctttctaatattcttaaaggctaaggagtcctttcaatataacagtaagataacttgtgatgttttagaagtaagcagaccattaatgtcaatgtggagtcttaatgttacatgaagttgatagtttctctgtgacccatttaaaaatacaaaccgagtagcatgcaattatgtaaagaaatatgaagattatatgtagtcacacattttctttagaattcttagtttggtgaaaacttgaatataaaggtattttgatttatatgacattttgatgatatttgaaaaaaaggaatttcctgacattttgcttttagatcatgtcccccattgtgctgtaatttaagccaacttggttcagtgaatgccatcaccatttccattgagaatttaaaactcaccagtgtttaacatgcaggcttctgagggctcccggagaatcagaccttaagcccagttgatttacttctaacgtgaaacttcgagttcctgtatactttgctagataatttgtggtacatctaaagcttagtcttaagtggcttgtgtgtggattttattcaacattcttgttgctagggtagagagaaatgttgctgagtagaaacaagagtacccagttcaatgtggtacagagagcagtccctaaaatctgtacacagtgtaatggaccactttaggagtcaagaggctgatttttcctatgaaattacattgcaacaggaagccttctagtatagttccttttactgttagaatatgtttttatgcatacgctatagctgctttcccatcttccaacaacaggtatcaggatgtaagcaagctttaaacagtgtgaagatggcaggatagtgtcatcggtaacagtcctctgactctaaatgtagttgctctgtaacactttgtgaatataacatcacaattctcatgtccttggggggggggggcatacccagtattagtatgttttagtgactaagcaatcatttttctgtttactcatgtacattttctctttaaaactaaaacctgtactgtgtatgtctccaaagccttttagcttagtttttaggaaatgaacactgaatggatcactttttagtgtagcaggtatgggatatgtgcattatagagagaccttgtcagctctctgggcctatttgaatgtttattgttggtgtgaggatggtaggggaatcagtaaatacaagttacgttggtttagcagagcaagctcagtgtgggtatttctctttgaagcgtggtgcgtgacgcactgtgagtagagaatttggtcaccctttgagtcctcttgcattttgcaaacttgctcagcaaatgcgtacctaccttgccccctaggtaaaagcaggaactactactgatttatctgtcactcagctgtctttatatgtgtgcttctgtgacttgtatcacacaagaatcttaaagatttcacaaattgttaccttttagctctgaatgttgagtattctggtgggctaacaacaagacaaactcttgacagtcatttgagaattttcatgaaacatttagctgaaaacattttataatttatgaaaaaaatgtgttaccttaaacttttacatatgtgggagacattaactgccatatttgagcatactgaattttaaatttaaaataaagctgcatatttttaaatgaaatgtttaacaaggattcatattttttgttttttaagattaaaaataatttatgtcttctcatgtggaacctcatctgtcacaatggttagattatacagaatggagcaaggcttgtagtggtttagcttacagtaaaattcttaatgtttagatgtgtttacttactggctgttatgtatacttttgagattttccacctgttctgtgtagttttctaaatgatactcctacttaaaaacagcattttagtatctattttctgtctccattaaatggtcctcattttctattgagtttggaagtgtgcacattgtgtgtgtgtgtgtgtgtgtgtgtgtgcacacgtgtgcgcgcccgtgcgtgtgtctatttgtggagtttgtatgggagaattagttttgaaagtgctagaatagagatgaaatttggttcaagtaaaattttcccactgggattttacagtttattgtaataaaatgttaattttggatgaccttgaatattaatgaatttgttagcctcttgatgtgtgcattaatgagatatatcaaagttgtatattaaaccaaagttggagttgtggaagtgtttttatgaagttccgtttggctaccaatggacataagactagaaataccttcctgtggagaatatttttcctttaaacaattaaaaaggttcattatttttgaaaaaaaaaaaaaaaaaa

SEQ ID NO: 4 - DNA, 1212 bp, Rattus norvegicus
atgacagccatcatcaaagagatcgttagcagaaacaaaaggagatatcaagaggatggattcgacttagacttgacctatatttatccaaatattattgctatgggatttcctgcagaaagacttgaaggtgtatacaggaacaatattgatgatgtagtaaggtttttggattcaaagcataaaaaccattacaagatatacaatctatgtgctgagagacattatgacaccgccaaatttaactgcagagttgcacagtatccttttgaagaccataacccaccacagctagaacttatcaaacccttttgtgaagatcttgaccaatggctaagtgaagacgacaatcatgttgcagcaattcactgtaaagctgggaaaggacggactggtgtaatgatttgtgcatatttattgcatcggggcaagtttttaaaggcacaagaggccctggatttttatggggaagtaaggaccagagataaaaagggagtaactattcccagtcagaggcgctatgtatattattatagctacctgttaaagaatcacctggattacagaccagtggcactgttgtttcacaagatgatgtttgaaactattccaatgttcagtggcggaacttgcaatccccagtttgtggtctgccagctaaaggtgaagatctactcctccaactcaggacccacgcggcgggaggacaagctcatgtactttgagttccctcagccattgcctgtgtgtggtgacatcaaagtagagttcttccacaaacagaacaagatgctcaaaaaggacaaaatgtttcacttttgggtaaatacgttcttcataccaggaccagaggaaacctcagaaaaagtggaaaatggaagtctttgtgatcaggaaatcgatagcatttgtagtatagagcgtgcggataatgacaaggagtatcttgtgctcaccctgacaaaaaatgatcttgacaaagcaaacaaagacaaggccaaccgatacttctctccaaattttaaggtgaagttatacttcacaaaaacagtagaggagccatcaaatccagaggctagcagttcaacttctgtgactccagacgttagtgacaatgaacctgatcattatagatattctgacaccactgactctgatccagagaatgaaccttttgatgaagatcagcattcacaaattacaaaagtctga

SEQ ID NO: 5 - DNA, 1396 bp, Canis familiaris
ccgcccgccgccaggcccggggccgcctgcagcctgcggaggaggccgcgccgcccgccgctcctgccgtctctctcctccttcctctccagccaccggctcccagacatgacagccatcatcaaggagatcgtcagcagaaacaaaaggcgctaccaggaggatgggttcgacttggacttgacctatatttatcccaacattattgctatggggtttcctgcagaaagacttgaaggcgtatacaggaacaatattgatgatgtagtaaggtttttggattcaaagcataaaaaccattacaagatatacaatctgtgtgctgaaagacattatgataccgccaaatttaactgcagagttgcacagtatccttttgaagaccataatccaccacagctagaacttatcaaacccttttgtgaagatcttgaccaatggctaagtgaagatgacaatcatgttgcagcaattcactgtaaagctggaaagggacgaactggtgtaatgatttgtgcatatttattacatcggggcaaatttctaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatctattcctccaattcaggacccacacgacgggaagacaagttcatgtactttgagttccctcagccattgcctgtgtgcggtgacatcaaagtagagttcttccacaaacagaacaagatgctaaaaaaggacaaaatgtttcacttttgggtaaacacattcttcataccaggaccagaggaaacctcagaaaaagtagaaaatggaagtctatgtgatcaagaaattgatagtatttgcagtatagaacgtgcagataatgacaaggaatatctagtactcactttaacaaaaaatgatctcgacaaagcaaataaagacaaggccaaccgatatttttctccaaattttaaggtgaagctgtacttcacaaaaactgtagaggagccatcaaacccggaggctagcagttcaacttctgtgacgccagatgttagtgacaatgaacctgatcattatagatattctgacaccactgactctgacccagagaatgaaccctttgatgaagatcagcacacacaaatcacaaaagtctgaattttttttaatcaagagggataaaacaccatgaaaacaaacttgaataaactgaaattggaccttttttttttaa

SEQ ID NO: 6 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 7 - DNA, 2239 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggtcctaataaatcttcactgtctgactttagtctcccactaaaactgcatttcctttctacaatttcaatttctccctttgcttcaaataaagtcctgacactattcatttga

SEQ ID NO: 8 - DNA, 1595 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacaggtttgagctgaattatcacatgaatataaatgggaaatcagtgttttagagagagaacttttcgacatatttcctgttcccttggaataaaaacatttcttctgaaattttaccgttaaaaaaaaaaaaaaaaaaaaaaaaa

SEQ ID NO: 9 - DNA, 2865 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggccaggaaatgagagtctcaaagccatgttattctgcctttttaaactatcatcctgtaatcaaagtaatgatggcagcgtgtcccaccagagcgggagcccagctgctcaggagtcatgcttaggatggatcccttctcttctgccgtcagagtttcagctgggttggggtggatgcagccacctccatgcctggccttctgcatctgtgatcatcacggcctcctcctgccactgagcctcatgccttcacgtgtctgttccccccgcttttcctttctgccacccctgcacgtgggccgccaggttcccaagagtatcctacccatttccttccttccactccctttgccagtgcctctcaccccaactagtagctaaccatcacccccaggactgacctcttcctcctcgctgccagatgattgttcaaagcacagaatttgtcagaaacctgcagggactccatgctgccagccttctccgtaattagcatggccccagtccatgcttctagccttggttccttctgcccctctgtttgaaattctagagccagctgtgggacaattatctgtgtcaaaagccagatgtgaaaacatctcaataacaaactggctgctttgttcaatgctagaacaacgcctgtcacagagtagaaactcaaaaatatttgctgagtgaatgaacaaatgaataaatgcataataaataattaaccaccaatccaacatccaga

SEQ ID NO: 10 - DNA, 5983 bp, Mus musculus
ctcccccagtcccgacccgagctaactagacgtctgggcagccccagcgcaacgcgcagcagcctccctcctcttcttcccgcactgtgcgctcctcctgggctagggcgtctggatcgagtcccggaggctaccgcctcccagacagacgacaggtcacctggacgcgagcctgtgtccgggtctcgtcgttgccggcgcagtcactgggcacaaccgtgggactccgtctgtctcggattaatcccggagagccagagccaacctctcccggtcagagatgcgaccctcagggaccgcgagaaccacactgctggtgttgctgaccgcgctctgcgccgcaggtggggcgttggaggaaaagaaagtctgccaaggcacaagtaacaggctcacccaactgggcacttttgaagaccactttctgagcctgcagaggatgtacaacaactgtgaagtggtccttgggaacttggaaattacctatgtgcaaaggaattacgacctttccttcttaaagaccatccaggaggtggccggctatgtcctcattgccctcaacaccgtggagagaatccctttggagaacctgcagatcatcaggggaaatgctctttatgaaaacacctatgccttagccatcctgtccaactatgggacaaacagaactgggcttagggaactgcccatgcggaacttacaggaaatcctgattggtgctgtgcgattcagcaacaaccccatcctctgcaatatggatactatccagtggagggacatcgtccaaaacgtctttatgagcaacatgtcaatggacttacagagccatccgagcagttgccccaaatgtgatccaagctgtcccaatggaagctgctggggaggaggagaggagaactgccagaaattgaccaaaatcatctgtgcccagcaatgttcccatcgctgtcgtggcaggtcccccagtgactgctgccacaaccaatgtgctgcggggtgtacagggccccgagagagtgactgtctggtctgccaaaagttccaagatgaggccacatgcaaagacacctgcccaccactcatgctgtacaaccccaccacctatcagatggatgtcaaccctgaagggaagtacagctttggtgccacctgtgtgaagaagtgcccccgaaactacgtggtgacagatcatggctcatgtgtccgagcctgtgggcctgactactacgaagtggaagaagatggcatccgcaagtgtaaaaaatgtgatgggccctgtcgcaaagtttgtaatggcataggcattggtgaatttaaagacacactctccataaatgctacaaacatcaaacacttcaaatactgcactgccatcagcggggaccttcacatcctgccagtggcctttaagggggattctttcacgcgcactcctcctctagacccacgagaactagaaattctaaaaaccgtaaaggaaataacaggctttttgctgattcaggcttggcctgataactggactgacctccatgctttcgagaacctagaaataatacgtggcagaacaaagcaacatggtcagttttctttggcggtcgttggcctgaacatcacatcactggggctgcgttccctcaaggagatcagtgatggggatgtgatcatttctggaaaccgaaatttgtgctacgcaaacacaataaactggaaaaaactcttcgggacacccaatcagaaaaccaaaatcatgaacaacagagctgagaaagactgcaaggccgtgaaccacgtctgcaatcctttatgctcctcggaaggctgctggggccctgagcccagggactgtgtctcctgccagaatgtgagcagaggcagggagtgcgtggagaaatgcaacatcctggagggggaaccaagggagtttgtggaaaattctgaatgcatccagtgccatccagaatgtctgccccaggccatgaacatcacctgtacaggcaggggaccagacaactgcatccagtgtgcccactacattgatggcccacactgtgtcaagacctgcccagctggcatcatgggagagaacaacactctggtctggaagtatgcagatgccaataatgtctgccacctatgccacgccaactgtacctatggatgtgctgggccaggtcttcaaggatgtgaagtgtggccatctgggccaaagataccatctattgccactgggattgtgggtggcctcctcttcatagtggtggtggcccttgggattggcctattcatgcgaagacgtcacattgttcgaaagcgtacactacgccgcctgcttcaagagagagagctcgtggaacctctcacacccagcggagaagctccaaaccaagcccacttgaggatattaaaggaaacagaattcaaaaagatcaaagttctgggttcgggagcatttggcacagtgtataagggtctctggatcccagaaggtgagaaagtaaaaatcccggtggccatcaaggagttaagagaagccacatctccaaaagccaacaaagaaatccttgacgaagcctatgtgatggctagtgtggacaaccctcatgtatgccgcctcctgggcatctgtctgacctccactgtccagctcattacacagctcatgccctacggttgcctcctggactacgtccgagaacacaaggacaacattggctcccagtacctcctcaactggtgtgtgcagattgcaaagggcatgaactacctggaagatcggcgtttggtgcaccgtgacttggcagccaggaatgtactggtgaagacaccacagcatgtcaagatcacagattttgggctggccaaactgcttggtgctgaagagaaagaatatcatgccgaggggggcaaagtgcctatcaagtggatggctttggaatcaattttacaccgaatttatacacaccaaagtgatgtctggagctatggtgtcactgtgtgggaactgatgacctttgggtccaagccttatgatggaatcccagcaagtgacatctcatccatcctagagaaaggagagcgccttccacagccacctatctgcaccatcgatgtctacatgatcatggtcaagtgctggatgatagatgctgatagccgcccaaagttccgagagttgattcttgaattctccaaaatggcccgagacccacagcgctaccttgttatccagggggatgaaagaatgcatttgccaagccctacagactccaacttttaccgagccctgatggatgaagaggacatggaggatgtagttgatgctgatgagtatcttatcccacagcaaggcttcttcaacagcccgtccacgtcgaggactcccctcttgagttctctgagtgcaactagcaacaattccactgtggcttgcattaatagaaatgggagctgccgtgtcaaagaagacgccttcttgcagcggtacagctccgaccccacaggtgctgtaacagaggacaacatagatgacgcattcctccctgtacctgaatatgtaaaccaatctgttcccaagaggccagcaggctctgtgcagaaccctgtctatcacaatcagcccctgcatccagctcctggaagagacctgcattatcaaaatccccacagcaatgcagtgggcaaccctgagtatctcaacactgcccagcctacctgtctcagtagtgggtttaacagccctgcactctggatccagaaaggcagtcaccaaatgagcctagacaaccctgactaccagcaggacttcttccccaaggaaaccaagccaaatggcatatttaagggccccacagctgaaaatgcagagtacctacgggtggcacctccaagcagtgagtttattggagcatgacaagaaggggcatcataccagctataaaatgtctggactttctagaatcccaggaccaactatggcagcacctccacttctggtagccatgcccacgctgtgtcaaatgtcactcagactggctttaaagcataactctgatgggctttgtcactgagccaagaagtgggcctctctcctgatgcactttgggaagttgaaggtacatcaattgatcttcgaactgtgaagattccacaaaaaaggtatccatcgagaacattgtccattggaacagaagtttgcctcatggtgaggtacatatgggaaaaaaacagacatatggagcttatatttagggaactttgggattcttgtctttattgatttgattgatgcactcttgtagtctggtacacagagttgcctggagccaactgaccagacagttggttccaccagctctgcatcaagacacttccgtggcaagacaactaaatgtataagaagtccatggatgccctgagcaggccacacttgtacagcattaaaccatggcagatacaataggataagccactttgttacttactggggctgggagaagaggaatgacggggtagaattttccctcagacgtactttttatataaatatgtccctggcacctaacacgcgctagtttaccagtgttttctattagacttccttctatgttttctgtttcattgttttgagttgtaaatatgtgttcctgtcttcatttcatgaagtaaacaaacaaacaaaaaacccagtattaagtattatcaaagaacaaccatgattccacattcgaacccattcaaaccatcagtattgtgaccaaaagcctttaactaagaaggagtaaccatgcaaaaatccatagaggaatttaacccaaaattttagtctcagcattgtgtctgctgaggtgtgtatatgagactacgaaagtgaactactcttcaaatccactttgccttcactcctctataccctaaatctagtgtaaaccacacatggaggataacttttttttttaattttaaaagtgtttattagatatgtttttcttcctggtaaactgcagccaaacatcagttaagagccatttttgataaacactatcacaatgatctcgggatccatcctttccgatttaccaagtgatggatagacgtgaactcataaacactacccataagacaaaacaatgagtgccagacaagacatcagccaggcaccagagcacagagcaggactgggcaatctgttggagatatctagaaagttcacaaaggaaacaagattgtccactaccttgtgagatctagcagtcataaataccagggaaatggaaagtgtgtttccttacagcaccaggtcttcgatcttcctaatgctgtgaccctttaatacagtttgccatgttgtggtgacccccaaccataaaattatttttgttgctacttcataactgtaaatttgctactcttacagaccacaatgtaaatatctgatatgctatctgatatgcaggctatctgacagaggtcgcaacccgcaggttgagagccactgccttcaaggctttaatcaagagagtagtgagctgagggctttactggtaagtcaggggcaagtccaactcaatcatcctcacatactggctgctccctcaggcctgagaatgaggcttgcagcatcctctggtttcctaaccgttatccatccctgactctcatctctgaaaatagatgtcatccatgaaattaaggagtgagaatattaagcagcatttatagagctcaaaattccatgtcatcaccaggaagtgccatgttgatcacagagaacacagaggagacatatagacagggttttgctcaaaattgggatatagaatgagcctgtcaggtacctatcaggagcggtaatccgtgagagagaaccgttgcaagccactctaactgtagcaatgaaaccctagtatttttgtactttgaaatactttcttataacaaaataaagtagcaaaaaaactgttcaaaaaaaaaaaaaaaaaaa

SEQ ID NO: 11 - DNA, 2678 bp, Mus musculus
ctcccccagtcccgacccgagctaactagacgtctgggcagccccagcgcaacgcgcagcagcctccctcctcttcttcccgcactgtgcgctcctcctgggctagggcgtctggatcgagtcccggaggctaccgcctcccagacagacgacaggtcacctggacgcgagcctgtgtccgggtctcgtcgttgccggcgcagtcactgggcacaaccgtgggactccgtctgtctcggattaatcccggagagccagagccaacctctcccggtcagagatgcgaccctcagggaccgcgagaaccacactgctggtgttgctgaccgcgctctgcgccgcaggtggggcgttggaggaaaagaaagtctgccaaggcacaagtaacaggctcacccaactgggcacttttgaagaccactttctgagcctgcagaggatgtacaacaactgtgaagtggtccttgggaacttggaaattacctatgtgcaaaggaattacgacctttccttcttaaagaccatccaggaggtggccggctatgtcctcattgccctcaacaccgtggagagaatccctttggagaacctgcagatcatcaggggaaatgctctttatgaaaacacctatgccttagccatcctgtccaactatgggacaaacagaactgggcttagggaactgcccatgcggaacttacaggaaatcctgattggtgctgtgcgattcagcaacaaccccatcctctgcaatatggatactatccagtggagggacatcgtccaaaacgtctttatgagcaacatgtcaatggacttacagagccatccgagcagttgccccaaatgtgatccaagctgtcccaatggaagctgctggggaggaggagaggagaactgccagaaattgaccaaaatcatctgtgcccagcaatgttcccatcgctgtcgtggcaggtcccccagtgactgctgccacaaccaatgtgctgcggggtgtacagggccccgagagagtgactgtctggtctgccaaaagttccaagatgaggccacatgcaaagacacctgcccaccactcatgctgtacaaccccaccacctatcagatggatgtcaaccctgaagggaagtacagctttggtgccacctgtgtgaagaagtgcccccgaaactacgtggtgacagatcatggctcatgtgtccgagcctgtgggcctgactactacgaagtggaagaagatggcatccgcaagtgtaaaaaatgtgatgggccctgtcgcaaagtttgtaatggcataggcattggtgaatttaaagacacactctccataaatgctacaaacatcaaacacttcaaatactgcactgccatcagcggggaccttcacatcctgccagtggcctttaagggggattctttcacgcgcactcctcctctagacccacgagaactagaaattctaaaaaccgtaaaggaaataacaggctttttgctgattcaggcttggcctgataactggactgacctccatgctttcgagaacctagaaataatacgtggcagaacaaagcaacatggtcagttttctttggcggtcgttggcctgaacatcacatcactggggctgcgttccctcaaggagatcagtgatggggatgtgatcatttctggaaaccgaaatttgtgctacgcaaacacaataaactggaaaaaactcttcgggacacccaatcagaaaaccaaaatcatgaacaacagagctgagaaagactgcaaggccgtgaaccacgtctgcaatcctttatgctcctcggaaggctgctggggccctgagcccagggactgtgtctcctgccagaatgtgagcagaggcagggagtgcgtggagaaatgcaacatcctggagggggaaccaagggagtttgtggaaaattctgaatgcatccagtgccatccagaatgtctgccccaggccatgaacatcacctgtacaggcaggggaccagacaactgcatccagtgtgcccactacattgatggcccacactgtgtcaagacctgcccagctggcatcatgggagagaacaacactctggtctggaagtatgcagatgccaataatgtctgccacctatgccacgccaactgtacctatggatgtgctgggccaggtcttcaaggatgtgaagtgtggccatctgggtacgttcaatggcagtggatcttaaagaccttttggatctaagaccagaagccatctctgactcccctctcaccttccagtttcttccaaatcctctgggccagccagaggtctcagattctgccctcttgccctgtgcccaccttgttgaccactggacagcatatgtgatggctactgctagtgccagcttcacaagaggttaacactacggactagccattcttcctatgtatctgtttctgcaaatacagccgctttacttaagtctcagcacttcttagtctcctcttttcctctcagtagcccaaggggtcatgtcacaaacatggtgtgaagggctactttgtcaaatgaaaaggtctatcttggggggcatttttttcttttctttttttcttgaaacacattgcccagcaaagccaataaatttctctcatcattttgtttctgataaattcttactattgat

SEQ ID NO: 12 - DNA, 4161 bp, Rattus norvegicus
ggaccgccaccaagacaggcgacgggtcacctggacgcgagtctgagtccgggtcccgtcgtcgttgccgacgcagtcaccgggcacgaccgtgggactcagtctgactcggattaatcccggagagccagagccaacgactgccgagccgggatgcgaccctcagggactgcgagaaccaagctactgctgctgctggctgcgctctgcgccgcaggtggggcgctggaggaaaagaaagtttgccaaggcacaagtaacaggctcacccaactaggcacctttgaagaccactttctgagcctccagaggatgttcaacaactgtgaagtggtccttggaaacttggaaatcacctatgtgcaaaggaattatgacctttccttcttaaagaccatccaggaggtggctggctatgttctcattgccctgaacaccgtggagagaatccctttggagaacctgcagatcatcaggggaaatgctctctacgaaaacacctacgccttagccgtcctgtccaactatggaaccaacaaaactgggcttagggaactgcccatgcggaacttacaggaaattctgatcggtgctgtgcgatttagcaacaaccccatcctctgcaatatggagaccatccagtggagggacatcgtccaagatgtctttctgagcaacatgtcaatggacgtacagcgccacctgacgggctgcccgaaatgtgatccgagctgtcccaatggaagctgctggggaagaggagaggagaactgccagaaattgaccaaaatcatctgcgcccagcaatgttcccggcgttgtcgtggcaggtcccctagcgactgctgccacaaccagtgtgccgcagggtgtacagggcccagagagagtgactgtctggtctgccacaggttccgagatgaagccacgtgcaaagacacctgcccaccactcatgctgtacaaccccaccacgtaccagatggatgtcaaccctgaggggaagtacagctttggtgccacctgtgtgaagaaatgccccagaaactacgtggtgacagatcacggctcgtgtgtccgggcctgtgggccagactactatgaagtagaagaagatggagtcagcaagtgtaaaaaatgtgacgggccctgccgcaaagtttgcaatggcataggcattggtgaatttaaagacacactctccataaatgctacaaacatcaaacacttcaagtactgcactgccatcagtggggacctccacatcctgccagtggcctttaagggggattctttcacccgcactcctcctctagacccacgggaactagaaattctcaaaactgtgaaggaaataacagggtttttgctgattcaggcttggcctgaaaactggactgacctccatgcttttgagaacctagaaataattcgtggcagaacaaagcaacatggtcagttttctctggcggttgtcggcctgaacataacatcgctggggttgcgttccctcaaggagatcagtgatggggatgtgattatttctgggaaccgaaatttgtgctacgcaaacactataaactggaaaaaactcttcgggacgcccaatcaaaagaccaaaatcatgaacaacagagctgaaaaggactgcaaggccacgaaccacgtctgtaatcctttatgctcctcggaaggctgctggggccctgagcccacggactgtgtctcctgccagaatgtgagcagaggcagggagtgcgtggacaagtgcaacatcctggagggggaaccgagggagtttgtggaaaattctgaatgcatccagtgccatccagaatgtctgccccagaccatgaacatcacctgtacaggccgggggccagacaactgcatcaagtgtgcccactatgttgatggtccccactgtgtcaagacctgcccttcgggcatcatgggggagaacaacaccctggtctggaagtttgcagatgccaataacgtctgccacctctgccatgcaaactgtacctatggatgtgctgggccaggccttaaaggatgtcaacaaccagaagggccaaagatcccatccatcgccactgggattgtgggtggcctcctcttcatagtagtggtggcccttgggatcggcctcttcatgcgtcgacgtcagcttgtccgaaaacgtacactacgccgcctgcttcaagagagagagctcgtggaacctctcacacccagcggagaagctccgaaccaagcccacttgaggatattaaaggaaacagaattcaaaaagatcaaagttctgggttcaggagcatttggcacagtgtataagggtctctggatcccagaaggcgagaaagtgaaaatccctgtggccatcaaggagttaagagaagccacatctcccaaagccaacaaggaaatccttgatgaagcctacgtgatggccagtgtggacaaccctcatgtatgccgcctcctgggcatctgtctgacctccactgtccagctcattacacaactcatgccctatggttgcctcctggactatgtccgagaacataaggacaacattggctcccagtacctactcaactggtgtgtgcagattgcaaagggcatgaactacctggaagaccggcgtttggtacaccgtgacttggcagccaggaatgtactggtaaagacaccacagcatgtcaagatcacagattttggactggccaaactgcttggtgctgaggagaaagaataccatgcagaggggggcaaagtgcctatcaagtggatggctttggaatcaattttacaccgaatttatacacaccaaagcgacgtctggagctatggagtcaccgtgtgggaactgatgacctttgggtccaagccttatgatgggatccctgcaagtgagatctcatccatcctagagaaaggagagcgccttccacagccacctatctgcaccatcgacgtctacatgatcatggtcaagtgctggatgatagatgctgatagccgcccaaagttccgagagttgattctcgaattctccaaaatggccagagacccacagcgctaccttgttatccagggggatgaaaggatgcatttgccgagccctacagactccaacttttaccgagccctgatggaggaggaggacatggaagacgtagttgatgctgatgaatacctcatcccacagcaaggcttcttcaacagcccatccacgtcacggactccactcttgagctctctgagtgcaaatagcaacagttccactgtggcttgcattaatagaaatgggagctgccgtgtcaaagaagacgccttcttgcaacggtatagctccgatcccaccagcgtcctgacagaggacaacatagatgacacattccttcccgtgcctgaatatataaaccaatctgttcccaagaggccggctggctctgtgcagaacccagtctatcacaatcagcccctgcatccagctcctggaagagacctgcattatcaaaatccccatagcaatgcggtgagcaaccctgagtatctcaacactgcccagccgacctgcctcagtagtgggtttgacagctctgccctctggatccagaaaggcagccaccaaatgagcctggacaaccctgactaccagcaggacttctttcccaaagaagccaagccgaatggcatctttaagggccccacagctgaaaatgcagagtacctgcgggtggcaccgccaagcagtgagtttagtggagcatgacattgaagaggcattgtaccagttacaaaccggactttccagaggcccaggaccaagccatggcagcacctctgctcctgacagccatgtccacattgtgtcaaatgtcaaaccctcagactggctttaaagcataactctgacgggctttgtcactgagccaagaagtgggcccctcccctgatgctctttgggaagttgaaggtatatctattggtcttcgaactgtgaagattccactgaaaggtatccatcgagaacattgtccttttggaacagaaggttgcctcatggtgaggtacatagggggaaaaaaaacagacctatggcgcttgctgacatagggaactctgggattcttgtctttattgatttgct

SEQ ID NO: 13 - DNA, 4788 bp, Canis familiaris
atgctttcaccacccaagtgtgagcaagtgcaaggccgtagaggcaccgcaggaccgggaaaccgggaagagcagcgggatggggttgtcctcctggggggtgtcgctgtgagcggaggccacacacgtggaattcccgagagcgccgaggaggagggagacctggagcagcgtccccgggtgggaaaagcaggggtgagaaaagcagtgggaggacgcagtcattgtctccactgtctgcagggctgcgaggtcgaggagaagcaggtcgcatctctgttcacccggaggaagtcatggggggacaagcttggggcccctaaagcacagggtcatgggaaccctgatcctatacttccagacctcctgttaggccttcctctgaagcagagctcggtgaagtacagcccgtggttcaaatccagcgactgcctgttttttaagccaagttttcgggcacgagccggtgccaggcccattcgtttggttcttgtctgtggctgctttttgcacgctggcggcggagcggaggggctgggacggacccttggagaatgttctgagcagcaagctgggagttctcctgcgggaattctgaaaggcccatccgaggtgtggccccctcagctttcagtgcccggcttgccccccccacccccgctcaaatccacccttgccccccgaggctccccgtggaagggctctgatatctgcctgcacctcattgccaggcttgcctgcgctcctgcaacaggcacctttggtcttggggcagctgaggctcgtaggctagctgaggggctggacaagccttacctggcaggctgtaaaacagttctggaaggtcaggattattttcagcatgcctacgacttgaggaaagcgtgccggggagagcttagagaagtcattcggggtcacgtcgtggcttattgtcgcaaccctgcacctacatcagtggtggcatcgttagggaccttgggattgggcaacgagtctggccccatgtgcatgcgttgccgccagctgtgtacagatgttccaagtgaagggtgcaatcagagcagcctgtgcttccgagtgcgagcagcccagagctgtgaggccacagaagagggaaatccccatagagttcaagaggatgtggcgattgcttcatatttaatgtcagtgtcagtgcatggggttgatgatgtggtgtctgaacatgtgctcgctgccgtggcaccaaaacgcaagggcagacggtcagtctgccaaggcacgagtaaccggctcacccagttgggcacttttgaagaccactttctgagccttcagaggatgttcaataactgtgaggtggtcctggggaatttggaaattacctacatgcaaaggaattatgacctttccttcttaaagaccatccaggaagttgccgggtatgtattgattgccctcaacacggtggagaagattcctttggaaaacctgcagatcatccgaggaaatgtgctctatgaaaacacccatgccttatccgtcctctccaactatggctcaaataaaactggactgcaggagcttcccctgagaaacttgcatgaaatactacaaggcgctgtgcgcttcagcaataaccctgtcctctgcaacgtggaaaccatccagtggcgggacatcgttgacaacgattttataagcaacatgtcgatggacatccagaaccaagcgggcaggtgccaaaagtgtgatccaagctgtcccaatggaagttgctggggtccgggaaaggagaattgccagaaactgaccaaaatcatctgtgcccagcagtgttcggggcgctgccgcggcaggtcccccagcgactgctgccacaaccagtgtgccgccggctgcacggggccccgcgagagcgactgcctggtctgccgcaagttccgggatgaagccacgtgcaaggacacctgcccgcccctcatgctctacaaccccaccacctaccaaatggacgtcaacccagagggaaaatacagctttggtgccacctgcgtgaagaaatgcccccgcaactacgtggtgacagaccacggttcatgtgtccgcgcctgcagctctgacagctacgaggtggaggaggatggtgtccgcaagtgtaagaagtgtgaggggccttgtcgcaaagtttgtaatggaatagggattggagagttcaaagacacactttccataaatgctacaaacattaaacacttcaaaaactgcacgtcgatcagtggagaccttcatatcttgccagtcgcatttagaggtgactccttcacgcataccctacctctagatccgaaggagctggatatcctgaaaactgtcaaggaaataacagggtttttgctgattcaggcctggcccgaaaataggactgacctccatgctttcgagaacctagaaatcatacgtggaagaacaaagcaacatggccagttttctctggcggtcgtcggcctgaacataacgtccttgggattacgctccctcaaggagataagcgatggagatgtgatcatctctggaaaccgaaaattgtgctatgcaaatacaataaactggaagaaactctttgggacttcaagtcagaaaaccaaaattataaacaacaaagatgaaaaagcctgcaaggccataggccacgtctgccatccattgtgctcctcagaaggctgctggggcccagggccgagagactgcgtgtcctgccgaaacgtcagccgtggcaaggaatgtgtggaaaagtgcaacattctggagggggagccaagggagtttgtggagaattcggagtgcatacagtgccatccggaatgcctgccccaggccatgaacataacctgcacaggacgggggccggacagctgcatcaagtgcgcccactacatcgatggccctcactgcgtcaagacctgcccggctggcatcatgggagaaaacaacaccctggtctggaagttttcggatggcagccgcatgtgccacctgtgccatccaaactgcacctatggctgtgagggaccaggtcttgaaggctgtgcaaaacctgggcccaagatcccatccattgctaccgggattgtcggcggcctcctcttggtggtggtggtggcccttggagtgggcctctttttgcgccggcgccacattgtccggaagcgcacgcttcgcagactgctgcaagaaagagagcttgtcgagcctcttacacccagcggagaagctcccaaccaggctctcttgaggatcttaaaggagacggagttcaaaaagatcaaggtgctgggctctggagcattcggcacagtgtacaagggactctggatcccagaaggcgagaaggttaaaattcccgtggccatcaaggaattgagagaagccacgtctcccaaagccaacaaggaaattcttgatgaagcctacgtgatggccagtgtggacaatccccacgtgtgccgcctcctgggcatctgcctgacgtccacggtgcagctcatcacgcagctcatgccctttggctgcctcctggactatgtccgcgagcacaaggacaacatcggctcccagcacctgctcaactggtgtgtgcagattgcaaagggcatgaactacctggaagaccggcgcttggtgcaccgcgacctggcggccaggaacgtcctggtgaagaccccgcagcacgtcaagatcacagattttgggctggccaaactgctgggtgccgaggagaaagagtaccacgcggaaggaggcaaagtgcctatcaagtggatggctttagaatcgattttacaccgaatttatacccaccaaagcgatgtgtggagctacggcgtcaccgtgtgggagctgatgaccttcgggtccaagccttacgacggtatccctgcaagtgagatctccaccatcctggagaagggagagcgcctcccgcagccgcccatatgcaccatcgatgtctacatgatcatggtcaagtgctggatgatagatgcagacagtcgcccaaaattccgtgagttgatcatcgaattctccaaaatggcccgagacccgcagcgctaccttgtcatccagggagatgagaggatgcatttgccaagccctacagactccaatttttaccgcgccctgatggacgaggaggacatggaggatgttgtggatgctgacgagtacctcatcccccagcagggcttcttccacagcccctccacttcccggacccccctcctaagttctctgagcgccaccagcaacagttccaacgtggcttgcatcgaccgaaatgggacctgtcccctcaaagaagacagcttcttgcagcggtacagctcagaccccactggcaccttgacggaggacaacatagatgacactttcctcccagcacccgaatacataaaccagtctgttcccaaaaggcctgcgggttctgtccagaatcccgtctatcacaatcagcctctaaatccagctcctgccagagaccctcactaccaaaatccccacagcaacgcagtggacaaccctgagtatctcaacacccaccccacctgcgtcaacagtgtcctcgacaggcccagcctctggacccaggaggccaaccaccaaatcagcctggacaaccctgactaccagcaggacttctttcccaaggaagccaagtccaatggcatttttaagggccctgcagctgaaaatgcagactacctgagggtagcgccaccaagcagtgagtttattggagcgtga